(19) United States
(12) Patent Application Publication
(10) Pub. No.: US 2004/0097718 A1
(43) Pub. Date: May 20, 2004

(54) Complete nucleotide sequence of staphlococcus aureus ribosomal protein gene, s20 and methods for the identification of antibacterial substances

(76) Inventors: James D. Pearson, Portage, MI (US); Jerry L. Slightom, Kalamazoo, MI (US); John G. Chosay, Kalamazoo, MI (US); Mark C. McCroskey, Kalamazoo, MI (US); Dean L. Shinabarger, Portage, MI (US); Sheri K Wilcox, Longmont, CO (US)

Correspondence Address: Edward F Rehberg, Pharmacia & Upjohn Company, Global Intellectual Property, 301 Henrietta Street, Kalamazoo, MI, 49001, US

(21) Appl. No.: 10/332,964

(22) PCT Filed: Jul. 19, 2001
(86) PCT No.: PCT/US01/21103

(51) Int. Cl. C07H 21/02; C07H 21/04

(52) U.S. Cl. 536/23.1

(57) ABSTRACT

The invention provides an isolated S. aureus ribosomal polypeptide S20, and the isolated polynucleotide molecules that encode them, vectors and host cells comprising such polynucleotide molecules and also methods for the identification of agents that effect ribosomal assembly.

### CROSS REFERENCE TO RELATED APPLICATIONS The present application claims priority of Application Serial No. 60/219,361 filed 19 Jul. 2000 which is hereby incorporated by reference.

### FIELD OF THE INVENTION

The present invention provides an isolated S. aureus S20 ribosomal polypeptide, and the isolated polynucleotide molecules that encode them, as well as vectors and host cells comprising such polynucleotide molecules. The invention also provides methods for the identification of agents that effect ribosomal assembly.

### BACKGROUND

The staphylococci, of which Staphylococcus aureus is the most important human pathogen, are hardy, gram-positive bacteria that colonize the skin of most humans. Staphylococcal strains that produce coagulase are designated S. aureus other clinically important coagulase-negative staphylococci are S. epidermidis and S. saprophyticus. When the skin or mucous membrane barriers are disrupted, staphylococci can cause localized and superficial infections that are commonly harmless and self-limiting. However, when staphylococci invade the lymphatics and the blood, potentially serious complications may result, such as bacteremia, septic shock, and serous metastatic infections, including endocarditis, arthritis, osteomyelitis, pneumonia and abscesses in virtually any organ. Certain strains of S. aureus produce toxins that cause skin rashes, food poisoning, or multisystem dysfunction (as in toxic shock syndrome). S. aureus and S. epidermidis together have become the most common cause of nonsocomial non-urinary tract infection in U.S. hosptitals. They are the most frequently isolated pathogens in both primary and secondary bacteremias and in cutaneous and surgical wound infections. See generally Harrison's Principles of Internal Medicine, 13^{th} ed., Isselbacher et. al. eds. McGraw-Hill, New York (1994), particularly pages 611-617.

Transient colonization of the nose by S. aureus is seen in 70-90 percent of people, of which 20 to 30 percent carry the bacteria for relatively prolonged periods of time. Independent colonization of the perineal area occurs in 5-20 percent of people. Higher carriage rates of S. aureus have been documented in persons with atopic dermatitis, hospital employees, hospitalized patients, patients whose care requires frequent puncture of the skin, and intravenous drug abusers.

Infection by staphylococci usually results from a combination of bacterial virulence factors and a diminution in host defenses. Important microbial factors include the ability of the staphylococcus to survive under harsh conditions, its cell wall constituents, the production of enzymes and toxins that promote tissue invasion, its capacity to persist intracellularly in certain phagocytes, and its potential to acquire resistance to antimicrobials. Important host factors include an intact mucocutaneous barrier, and adequate number of functional neutrophils, and removal of foreign bodies or dead tissue.

Once the skin or mucosa have been breached, local bacterial multiplication is accompanied by inflammation, neutrophil accumulation, tissue necrosis, thrombosis and fibrin deposition at the site of infection. Later, fibroblasts create a relatively avascular wall about the area. When host mechanisms fail to contain the cutaneous or submucosal infection, staphylococci may enter the lymphatics and the bloodstream. Common sites of metastatic spread include the lungs, kidneys, cardiac valves, myocardium, liver, spleen, bone and brain.

Antimicrobial resistance by staphylococci favors their peristence in the hospital environment. Over 90 percent of both hospital and community strains of S. aureus causing infection are resistant to penicillin. This resistance is due to the production of β lactamase enzymes. The genes for these enzymes are usually carried by plasmids. Infections due to organisms with such acquired resistance can sometimes be treated with β lactamase resistant penicillin derivatives. However the true penicillinase-resistant S. aureus organisms, called methicillin resistant S. aureus (MILSA), are resistant to all the β lactam antibiotics and the cephalosporins. MRSA resistance is chromosomally mediated and involves production of an altered penicillin-binding protein (PBP 2a or PBP 2′) with a low binding for B lactams. MRSA frequently also have acquired plasmids mediating resistance to erythromycin, tetraccyline, chloramphenicol, clindamycin, and aminoglyucosides. MRSA have become increasingly common worldwide, particularly in tertiary-care referral hospitals. In the United States, approximately 32 percent of hospital isolates of S. aureus are methicillin resistant. Methicillin resistant staphylococci are a serious clinical and economic problem, since treatment of these infections often requires vancomycin, an antibiotic that is more difficult to administer and more expensive than the penicillins. Quinolone antimicrobial agents have been used to treat methicillin-resistant staphylococcal infections. Unfortunately, resistance to these antibiotics has also developed rapidly. Sixty to 70% of methicillin resistant S. aureus isolates are also quinolone resistant.

A pressing need exists for new chemical entities that are effective in the treatment of staphylococcal infections. One fruitful area of research has been in the area of agents which inhibit protein synthesis. A large number of antibacterial agents, including many in current clinical use, inhibit protein synthesis in bacteria by interfering with essential functions of the ribosome. When ribosomal function is perturbed, protein synthesis may cease entirely or, alternatively, it may be sufficiently slowed so as to stop normal cell growth and metabolism. Differences between the prokaryotic 70S ribosomes (composed of 50S and 30S subunits) and the eukaryotic 80S ribosome (composed of 60S and 40S subunits) underlie the basis for the selective toxicity of many antimicrobial agents of this class. However, a limited subset of this class of antimicrobial agents exhibits some cross-reactivity with the 70S ribosomes of eukaryotic mitochondria. This cross-reactivity probably accounts for the host cells cytotoxicity effects observed with some agents and has limited their use as clinical antimicrobial agents. Other agents (e.g., tetracycline), which affect the function of eukaryotic 80S ribosomes in vitro, are still used clinically to treat bacterial infections as the concentrations employed during antimicrobial therapy are not sufficient to elicit host cell toxicity side-effects.

Moreover, protein biosynthesis inhibitors can be divided into a number of different classes based on differences in their mechanisms of action. The aminoglycoside agents (e.g., streptomycin) bind irreversibly to the 30S subunit of the ribosome, thereby slowing protein synthesis and causing mis-translation (i.e., mis-reading) of the mRNA. The resulting errors in the fidelity of protein synthesis are bacteriocidal, and the selective toxicity of this family of agents is increased by the fact that bacteria actively transport them into the cell. The tetracycline family of agents (e.g., doxycycline) also binds to the 30S ribosome subunit, but does so reversibly. Such agents are bacteriostatic and act by interfering with the elongation phase of protein synthesis by inhibiting the transfer of the amino acid moieties of the aminoacyl-tRNA substrates into the growing polypeptide chain. However, inhibition mediated by the tetracyclines is readily reversible, with protein synthesis resuming once intracellular levels of the agent's decline. Chloramphenicol and the macrolide family of agents (e.g., erythromycin), in contrast, act on the function/activity of the 50S subunit of the ribosome. These agents are bacteriostatic in nature, and their effects are reversible. It has also been suggested that both chloramphenicol and the macrolides may have a second mode of action involved in ribosomal assembly. Champney and Burdine (1995). Finally, puromycin acts as a competitive inhibitor of the binding of aminoacyl-tRNA's to the so-called aminoacyl site (i.e., A-site) of the ribosome and acts as a chain-terminator of the elongation phase as a result of its incorporation into the growing peptide chain.

It has been shown in E. coli that mutants which lack S20 in ribosomes, as judged by 2-dimensional electrophoresis are impaired in 30S subunit association with 50S subunits to form 70S ribosomes. Ryden-Aulin et al. (1993) Molecular Microbiology 7(6) 983-992. The mutants described by Ryden-Aulin misread nonsense codons and show a greatly reduced growth rate. Because of this growth impairment S20 ribosomal polypeptide is an attractive molecular target for the development of antibacterial agents effective against S. aureus and related organisms. It has also been noted that mitochondrial ribosomes lack a homolog of the bacterial S20 protein. Koc et al. (2001) J. Biol. Chem 276 (22) 19363-19374. The lack of a mitochondrial counterpart makes S20 even more attractive as a bacteria-specific target.

This document discloses important new methods of identifying antibacterial substances related to the bacterial ribosomal assembly process, and to the Staphlylococoal ribosomal protein S20 and it for the first time discloses the full nucleotide and amino acid sequence of Staphylococcus aureus S20 ribosomal polypeptide

### Literature Cited

Patent Documents

U.S. Pat. No. 3,940,475

U.S. Pat. No. 5,843,669

U.S. Pat. No. 6,083,924

WO 97/09433, Cell-Cycle Checkpoint Genes

Non Patent Documents

1. Vartikar, J. V. and D. E. Draper, S4-16 S ribosomal RNA complex. Binding constant measurements and specific recognition of a 460-nucleotide region. J Mol Biol, 1989. 209(2): p. 221-34.

2. Altschul, S. F., et al., Basic local alignment search tool. J Mol Biol, 1990. 215(3): p. 403-10.

3. Ausubel, F. M., Current protocols in molecular biology. 1994, New York: John Wiley & Sons. 3 v. (loose-leaf).

4. Bolton, A. E. and W. M. Hunter, The labelling of proteins to high specific radioactivities by conjugation to a 125I-containing acylating agent. Biochem J, 1973. 133(3): p. 529-39.

5. Champney, W. S. and R. Burdine, Macrolide antibiotics inhibit 50S ribosomal subunit assembly in Bacillus subtilis and Staplhylococcus aureus. Antimicrob Agents Chemother, 1995. 39(9): p. 2141-4.

6. Clauser, K. R., et al, Rapid mass spectrometric peptide sequencing and mass matching for characterization of human melanoma proteins isolated by two-dimensional PAGE. Proc Natl Acad Sci USA, 1995. 92(11): p. 5072-6.

7. Creighton, T. E., Proteins: structures and molecular properties. 2nd ed. 1993, New York: W.H. Freeman. xiii, 507.

8. Devereux, J., P. Haeberli, and O. Smithies, A comprehensive set of sequence analysis programs for the VAX. Nucleic Acids Res, 1984. 12(1 Pt 1): p. 387-95.

9. Ducret, A., et al., Characterization of human serum amyloid A protein isoforms separated by two-dimensional electrophoresis by liquid chromatography/electrospray ionization tandem mass spectrometry. Electrophoresis, 1996. 17(5): p. 866-76.

10. Evan, G. I., et al., Isolation of monoclonal antibodies specific for human c-myc proton-oncogene product. Mol Cell Biol, 1985. 5(12): p. 3610-6.

11. Figeys, D., et al., Protein identification by capillary zone electrophoresis/microelectrospray ionization-tandem mass spectrometry at the subfemtomole level. Anal Chem, 1996. 68(11): p. 1822-8.

12. Gevaert, K., et al., Structural analysis and identification of gel-purified proteins, available in the femtomole range, using a novel computer program for peptide sequence assignment, by matrix-assisted laser desorption ionization-reflectron time-of-flight-mass spectroymietry. Electrophoresis, 1996. 17(5): p. 918-24.

13. Gribskov, M. R. and J. Devereux, Sequence analysis primer. 1991, New York Basingstroke, Hants, England: Stockton Press; Macmillan Publishers. xv, 279.

14. Griffin, A. M. and H. G. Griffin, Computer analysis of sequence data. 1994, Totowa, N.J.: Humana Press. 2 v.

15. Harrison, T. R. and K. J. Isselbacher, Harrison's principles of internal medicine. 13th/ed. 1994, New York: McGraw-Hill. 2 v. (xxxii, 2496, 154).

16. Heijne, G. v., Sequence analysis in molecular biology: treasure trove or trivial pursuit. 1987, San Diego: Academic Press. xii, 188.

17. Kohler, G. and C. Milstein, Continuous cultures of fused cells secreting antibody of predefined specificity. Nature, 1975. 256(5517): p. 495-7.

18. Lesk, A. M. and CODATA. Task Group on Coordination of Protein Sequence Data Banks., Computatioyial molecular biology: sources and methods for sequence analysis. 1988, Oxford; New York: Oxford University Press. xii, 254.

19. Lutz, W., et al., Internalization of vasopressin analogs in kidney and smooth muscle cells: evidence for receptor-mediated endocytosis in cells with V2 or V1 receptors. Proc Natl Acad Sci USA, 1990. 87(17): p. 6507-11.

20. Neidhardt, F. C., Escherichia coli and Salmonella typhiurium: cellular and molecular biology. 1987, Washington, D.C.: American Society for Microbiology. 2 v. Chapter 10

21. Nomura, M., A. Tissiéres, and P. Lengyel, Ribosomes. 1974, [Cold Spring Harbor, N.Y.]: Cold Spring Harbor Laboratory. pp. 193-223

22. Paborsky, L. R., et al., Mammalian cell transient expression of tissue factor for the production of antigen. Protein Eng, 1990. 3(6): p. 547-53.

23. Patterson, S. D. and R. Aebersold, Mass spectrometric approaches for the identification of gel-separated proteins. Electrophoresis, 1995. 16(10): p. 1791-814.

24. Reisfeld, R. A. and S. Sell, Monoclonal antibodies and cancer therapy: proceedings of the Roche-UCLA symposium held in Park City, Utah, Jan. 26-Feb. 2, 1985. 1985, New York: Liss. xxii, 609.

25. Ryden-Aulin, M., et al., Ribosome activity and modification of 16S RNA are influenced by deletion of ribosomal protein S20. Mol Microbiol, 1993. 7(6): p. 983-92.

26. Sambrook, J., E. F. Fritsch, and T. Maniatis, Molecular cloning: a laboratory manual. 2nd ed. 1989, Cold Spring Harbor, N.Y.: Cold Spring Harbor Laboratory. 3 v. in 1.

27. Skinner, R. H., et al., Use of the Glu-Glu-Phe C-terminal epitope for rapid purification of the catalytic domain of normal and mutant ras GTPase-activating proteins. J Biol Chem, 1991. 266(22): p. 14163-6.

28. Smith, D. W., Biocomputing: informatics and genome projects. 1994, San Diego: Academic Press. xii, 336.

29. Syvanen, J. M., Y. R. Yang, and M. W. Kirschiner, Preparation of 125 I-Catalytic subunit of asparatate transcarbamylase and its use in studies of the regulatory subunit. J Biol Chem, 1973.248(11): p.3762-8.

30. Held, W. A. and M. Nomura, Escherichia coli 30 S ribosomal proteins uniquely required for assembly. J Biol Chem, 1975.250(8): p.3179-84.

31. Koc, E. C., Burkhardt, W., Blackburn, K., Mosley, A., and Spremulli, L. L., The Small Subunit of the Mammalian Mitochondrial Ribosome, J. Biol. Chem., 2001 276 (22)19363-19374.

32. Wimberly B. T., Broderson, D. E., Clemmons, W. M., Morgan-Warren, R. J., Carter, A. P. Vonrhein, C., Hartsch, T., Ramikrishnan, Structure of the 30S ribosomal subunit. Nature 2000, 407; 327-338.

### BRIEF DESCRIPTION OF THE SEQUENCE LISTINGS

SEQ ID NO:1 Complete coding sequence of S20 ribosomal polypeptide

SEQ ID NO:2 Predicted polypeptide sequence of S20 ribosomal polypeptide

SEQ ID NO:3 Sequencing Primer

SEQ ID NO:4 Sequencing Primer

SEQ ID NO:5 Sequencing Primer

SEQ ID NO:6 Sequencing Primer

SEQ ID NO:7 Sequencing Primer SEQ ID NO:8 Sequencing Primer

SEQ ID NO:9 PCR Primer

SEQ ID NO:10 PCR Primer

SEQ ID NO:11 DNA sequence for Staphylococcus aureus S4 ribosomal protein gene (coding and flanking sequences)

SEQ ID NO:12 Polypeptide sequence for Staphylococcus aureus S4 ribosomal protein

SEQ ID NO:13 DNA sequence for Staphylococcus aureus S7 ribosomal protein gene (coding and flanking sequences)

SEQ ID NO:14 Polypeptide sequence for Staphylococcus aureus S7 ribosomal protein

SEQ ID NO:15 DNA sequence for Staphylococcus aureus S8 ribosomal protein gene (coding and flanking sequences)

SEQ ID NO:16 Polypeptide sequence for Staphylococcus aureus S8 ribosomal protein

SEQ ID NO:17 DNA sequence for Staphylococcus aureus S15 ribosomal protein gene (coding and flanking sequences)

SEQ ID NO:18 Polypeptide sequence for Staphylococcus aureus S15 ribosomal protein

SEQ ID NO:19 DNA sequence for Staphylococcus aureus S17 ribosomal protein gene (coding and flanking sequences)

SEQ ID NO:20 Polypeptide sequence for Staphylococcus aureus S17 ribosomal protein

SEQ ID NO:21 DNA sequence for Staphylococcus aureus 16S ribosomal RNA gene (coding and flanking sequences)

SEQ ID NO:22 DNA sequence for Staphylococcus aureus S1 ribosomal protein gene (coding and flanking sequences)

SEQ ID NO:23 Polypeptide sequence for Staphylococcus aureus S1 ribosomal protein gene

SEQ ID NO:24 DNA sequence for Staphylococcus aureus S2 ribosomal protein gene (coding and flanking sequences)

SEQ ID NO:25 Polypeptide sequence for Staphylococcus aureus S2 ribosomal protein

SEQ ID NO:26 DNA sequence for Staphylococcus aureus S3 ribosomal protein gene (coding and flanking sequences)

SEQ ID NO:27 Polypeptide sequence for Staphylococcus aureus S3 ribosomal protein

SEQ ID NO:28 DNA sequence for Staphylococcus aureus S5 ribosomal protein gene (coding and flanking sequences)

SEQ ID NO:29 Polypeptide sequence for Staphylococcus aureus S5 ribosomal protein

SEQ ID NO:30 DNA sequence for Staphylococcus aureus S6 ribosomal protein gene (coding and flanking sequences)

SEQ ID NO:31 Polypeptide sequence for Staphylococcus aureus S6 ribosomal protein

SEQ ID NO:32 DNA sequence for Staphylococcus aureus S9 ribosomal protein gene (coding and flanking sequences)

SEQ ID NO:33 Polypeptide sequence for Staphylococcus aureus S9 ribosomal protein

SEQ ID NO:34 DNA sequence for Staphylococcus aureus S10 ribosomal protein gene (coding and flanking sequences)

SEQ ID NO:35 Polypeptide sequence for Staphylococcus aureus S80 ribosomal protein

SEQ ID NO:36 DNA sequence for Staphylococcus aureus S11 ribosomal protein gene (coding and flanking sequences)

SEQ ID NO:37 Polypeptide sequence for Staphylococcus aureus S11 ribosomal protein

SEQ ID NO:38 DNA sequence for Staphylococcus aureus S12 ribosomal protein gene (coding and flanking sequences)

SEQ ID NO:39 Polypeptide sequence for Staphylococcus aureus S12 ribosomal protein

SEQ ID NO:40 DNA sequence for Staphylococcus aureus S13 ribosomal protein gene (coding and flanking sequences)

SEQ ID NO:41 Polypeptide sequence for Staphylococcus aureus S13 ribosomal protein

SEQ ID NO:42 DNA sequence for Staphylococcus aureus S14 ribosomal protein gene (coding and flanking sequences)

SEQ ID NO:43 Polypeptide sequence for Staphylococcus aureus S14 ribosomal protein

SEQ ID NO:44 DNA sequence for Staphylococcus aureus S16 ribosomal protein gene (coding and flanking sequences)

SEQ ID NO:45 Polypeptide sequence for Staphylococcus aureus S16 ribosomal protein

SEQ ID NO:46 DNA sequence for Staphylococcus aureus S18 ribosomal protein gene (coding and flanking sequences)

SEQ ID NO:47 Polypeptide sequence for Staphylococcus aureus S18 ribosomal protein

SEQ ID NO:48 DNA sequence for Staphylococcus aureus S19 ribosomal protein gene (coding and flanking sequences)

SEQ ID NO:49 Polypeptide sequence for Staphylococcus aureus S19 ribosomal protein

SEQ ID NO:50 DNA sequence for Staphylococcus aureus S20 ribosomal polypeptide gene (coding and flanking sequences)

SEQ ID NO:51 DNA sequence for Staphylococcus aureus S21 ribosomal protein gene (coding and flanking sequences)

SEQ ID NO:52 Polypeptide sequence for Staphylococcus aureus S21 ribosomal protein

SEQ ID NO:53 Exemplary S4 Forward PCR Primer

SEQ ID NO:54 Exemplary S4 Reverse PCR Primer

SEQ ID NO:55 Exemplary S18 Forward PCR Primer

SEQ ID NO:56 Exemplary S18 Reverse PCR Primer

SEQ ID NO:57 Exemplary S6 Forward PCR Primer

SEQ ID NO:58 Exemplary S6 Reverse PCR Primer

SEQ ID NO:59 Exemplary 16S H-44 Helical RNA Forward PCR Primer

SEQ ID NO:60 Exemplary 16S H-44 Helical RNA Reverse PCR Primer

SEQ ID NO:61 Exemplary 16S H-7, 8, 9, 10 & 11 Helical RNA Forward PCR Primer

SEQ ID NO:62 Exemplary 16S H-7, 8, 9, 10 & 11 Helical RNA Reverse PCR Primer

### BRIEF DESCRIPTION OF THE FIGURES

FIG. 1-DNA Coding Region and Amino Acid Sequence of the S20 ribosomal polypeptide

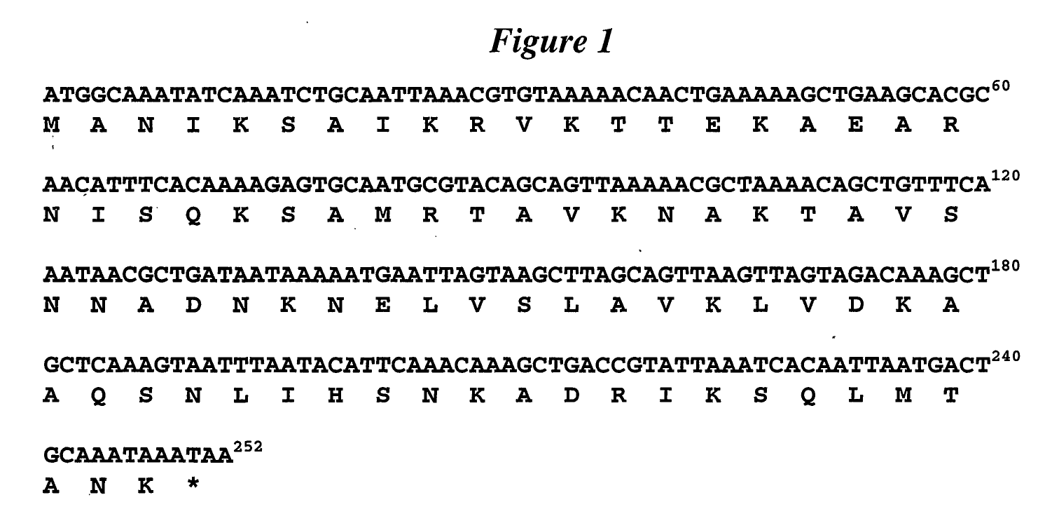

FIG. 2. Column Profile of HiPrep SPXL Column

FIG. 3. Coomassie-stained NuPage Gels of S20 ribosomal polypeptide fractions. Using Novex NuPage™ Bis-gels Tris (4-12%) with a MES Buffer system

FIG. 4 Graphic illustration of how specific inhibition of S20 ribosomal polypeptide binding to RNA is detected.

FIG. 5 Graphic illustration of a ribosomal assembly map incorporating direct binding S proteins (S4, S8, S7, S17, and S20) as well as some proteins which integrate themselves into ribosomes by reliance on protein-protein interactions (non-direct binding proteins) (S3, S5, S9, S10, S12, S14, S16 and S19). Arrows between proteins indicate the effect of a protein on another whose binding it enhances. Thick arrows indicate a principal contribution. Thin arrows indicate lesser contribution. Noller and Nomura (1987)

FIG. 6 Graphical illustration of a ribosomal assembly assay incorporating direct binding S proteins (S4, S8, S7, S17, and S20) as well as proteins which integrate themselves into ribosomes by reliance on protein-protein interactions “non direct binding proteins” (S3, S5, S9, S10, S12, S14, S16 and S19).

### SUMMARY OF THE INVENTION

The present invention provides an isolated S. aureus S20 ribosomal polypeptide, and the isolated polynucleotide molecules that encode them, as well as vectors and host cells comprising such polynucleotide molecules. The DNA sequences provided herein may be used in the discovery and development of antibacterial compounds. The encoded polypeptide, upon expression, can be used as a target for the screening of antibacterial drugs. High-throughput assays for identifying inhibitors of ribosomal assembly are provided. Solid phase high throughput assays are provided, as are related assay compositions, integrated systems for assay screening and other features that will be evident upon review.

In one embodiment, the invention provides an isolated S20 ribosomal polypeptide comprising the amino acid sequence set forth in SEQ ID NO: 2. The DNA and predicted amino acid sequence of Staphylococcus aureus S20 ribosomal polypeptide is displayed below:

ATGGCAAATATCAAATCTCCAATTAAACCTGTAAAAACAACTGAAAAAGCTGAAGCACGC⁶⁰
M A N I K S A I K R V K T T E K A E A R
AACATTTCACAAAAGAGTGCAATGCGTACAGCAGTTAAAAACGCTAAAACAGCTGTTTCA¹²⁰
N I S Q K S A M R T A V K N A K T A V S
AATAACGCTGATAATAAAAATGAATTAGTAAGCTTAGCAGTTAAGTTAGTAGACAAAGCT¹⁸⁰
N N A D N K N E L V S L A V K L V D K A
GCTCAAAGTAATTTAATACATTCAAACAAAGCTGACCGTATTAAATCACAATTAATGACT²⁴⁰
A Q S N L I H S N K A D R I K S Q L M T
CCAAATAAATAA²⁵²
A N K *

Although SEQ ID NOS:1 and 2 provide particular S. aureus sequences, the invention is intended to include within its scope other S. aureus allelic variants. Allelic variants are understood to mean naturally-occurring base changes in the species population which may or may not result in an amino acid change of the DNA sequences herein

The present invention also includes include variants of the aforementioned polypetide, that is polypeptides that vary from the referents by conservative amino acid substitutions, whereby a residue is substituted by another with like characteristics.

The nucleic acids of the invention include those nucleic acids coding for the same amino acids in the S20 ribosomal polypeptide due to the degeneracy of the genetic code

In another embodiment, the invention provides isolated polynucleotides (e.g. RNA and DNA, both naturally occurring and synthetically derived, both single and double stranded) that comprise a nucleotide sequence encoding the amino acid sequence of the polypeptides of the invention. Such polynucleotides are useful for recombinantly expressing the enzyme and also for detecting expression of the polypeptides in cells (e.g. using Northern hybridization and in situ hybridization assays). Specifically excluded from the definition of polynucleotides of the invention is the entire isolated chromosome of the native host cells. A preferred polynucleotide of the invention set forth in SEQ ID NO:1 corresponds to the naturally occurring S20 ribosomal polypeptide encoding nucleic acid sequence. It will be appreciated that numerous other sequences exist that also encode S20 ribosomal polypeptide of SEQ ID NO:2 due to the well known degeneracy of the universal genetic code. In another preferred embodiment the invention is directed to all isolated degenerate polynucleotides encoding the S20 ribosomal polypeptide.

In another embodiment the invention provides an isolated nucleic acid comprising the nucleotide sequence having least 60%, 70%, 80, 90% identity with SEQ ID NO:1. In one embodiment, the invention provides an isolated S20 ribosomal polypeptide comprising the amino acid sequence set forth in SEQ ID NO: 2.

In a related embodiment the invention provides vectors comprising a polynucleotide of the invention. Such vectors are useful, e.g. for amplifying the polynucleotides in host cells to create useful quantities thereof. In preferred embodiments, the vector is an expression vector wherein the polynucleotide of the invention is operatively linked to a polynucleotide comprising an expression control sequence. Such vectors are useful for recombinant production of polypeptides of the invention.

In another related embodiment, the invention provides host cells that are transformed with polynucleotides or vectors of the invention. As stated above, such host cells are useful for amplifying the polynucleotides and also for expressing the S20 ribosomal polypeptide or a fragment thereof encoded by the polynucleotide.

In still another related embodiment, the invention provides a method for producing the S20 ribosomal polypeptide (or a fragment thereof) comprising the steps of growing a host cell of the invention in a nutrient medium and isolating the S20 ribosomal polypeptide from the cells.

In still another related embodiment, the invention provides a method for testing for inhibitors of ribosomal assembly comprising the steps of contacting a labeled S20 ribosomal polypeptide with a ribosomal RNA in the presence and the absence of a test agent, determining the amount of S20 ribosomal polypeptide specifically bound to said RNA both in the presence of a test agent and in the absence of said test agent, and comparing the amount of protein determined in the presence of the test agent to the amount of protein determined in step in the absence of the test agent.

A decrease in the amount of protein determined in the presence of test agent compared to that determined in the absence of the test agent indicates that said agent is an inhibitor of ribosomal assembly

In still another related embodiment, the invention provides a method for testing for inhibitors of ribosomal assembly comprising the steps of contacting at least one direct binding ribosomal polypeptide selected from the group consisting of S4, S7, S8, S15, S17 and S20 with 16S ribosomal RNA in the presence and absence of a test agent and determining the amount of direct binding protein bound to the RNA in the presence of a test agent; and in the absence of said test agent; and comparing the amount direct binding protein determined under both sets of conditions. A decrease in the amount of direct binding protein determined in the presence of test agent compared to that determined in the absence of the test agent indicates that said agent is an inhibitor of ribosomal assembly

In still another related embodiment the invention provides a method for testing for inhibitors of ribosomal assembly comprising the steps of contacting at least one direct binding ribosomal polypeptide selected from the group consisting of S4, S7, S8, S15, S17 and S20 with 16S ribosomal RNA to form a polyribonucleotide protein complex and; contacting said polyribonucleotide protein complex with at least one non-direct binding ribosomal polypeptide selected from the group consisting of S1, S2, S3, S5, S6, S9, S10, S11, S12, S13, S14, S16, S18, S19, and S21. in the presence and absence of a test agent; and then determining the amount of at least one non-direct binding ribosomal polypeptide bound to the RNA in the presence and the absence of a test agent and then comparing the amount of least one non direct binding ribosomal polypeptide bound under both conditions

In still another related embodiment the invention provides an isolated S20 ribosomal polypeptide comprising an amino acid sequence at least 70%, 80, 90%, 95% identical to the sequence of SEQ ID NO:2.

In addition to the foregoing, the invention includes as an additional aspect, all embodiments of the invention narrower in scope in any way than the variations specifically mentioned above. Although the applicant(s) invented the full scope of the claims appended hereto, the claims appended are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### DETAILED DESCRIPTION OF THE INVENTION

The foregoing is provided to further facilitate understanding of the applicant's invention but is not intended to limit the scope of applicant's invention.

Definitions

As used hereinafter “Isolated” means altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

As used hereinafter “Polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

As used hereinafter “Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation prenylation, racemization, selenoylation, sulfation, transfer-RNA mediated addition of amino acids to proteins such as arginylation, and ubiquitination (see, for instance, Proteins-Structure and Molecular Properties, 2nd Ed., T. E. Creighton, W. H. Freeman and Company, New York, 1993; Wold, F., Post-translational Protein Modifications: Perspectives and Prospects, pgs. 1-12 in Postranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:4842).

As used hereinafter “Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

As used hereinafter “Identity” is a measure of the identity of nucleotide sequences or amino acid sequences. In general, the sequences are aligned so that the highest order match is obtained. “Identity” per se has an art-recognized meaning and can be calculated using published techniques (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J Applied Math (1988) 48:1073. Methods to determine identity and similarity are codified in computer programs. Preferred computer program methods to determine identity and similarity between two sequences include, but are not limited to, GCG program package (Devereux, J., et al., Nucleic Acids Research (1984) 12(1):387), BLASTP, BLASTN, and FASTA (Atschul, S. F. et al., J Molec Biol (1990) 215:403). The well known Smith Waterman algorithm may be used to determine identity. The Gap program (Wisconsin Sequence Analysis Package, Version 8 for Unix, Genetics Computer Group, University Research Park, Madison, Wis.) is one such program which uses the algorithm of Smith and Waterman (Adv. Appl. Math. 2:482-489 (1981)).

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 by the numerical percent of the respective percent identity (divided by 100) and subtracting that product from said total number of nucleotides in SEQ ID NO:1, or:

nₙ≦xₐ−(xₐ·y)

wherein n, is the number of nucleotide alterations, xₙ is the total number of nucleotides in SEQ ID NO:1, and y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the numerical percent of the respective percent identity (divided by 100) and then subtracting that product from said total number of amino acids in SEQ ID NO:2, or:

nₐ≦xₐ−(xa*y)

wherein nₐ is the number of amino acid alterations, xₐ is the total number of amino acids in SEQ ID NO:2, and y is, for instance 0.70 for 70%, 0.80 for 80%, 0.85 for 85% etc., and wherein any non-integer product of xₐ and y is rounded down to the nearest integer prior to subtracting it from xₐ. Identity has been similarly defined in U.S. Pat. No. 6,083,924 which is hereby incorporated by reference.

The present invention provides isolated polynucleotides (e.g., DNA sequences and RNA transcripts, both sense and complementary antisense strands, both single and double stranded) encoding a Staphylococcus aureus ribosomal protein S20. The nucleic acids of the invention include those nucleic acids coding for the same amino acids in the S20 ribosomal polypeptide due to the degeneracy of the genetic code. DNA polynucleotides of the invention include genomic DNA and DNA that has been synthesized in whole or in part. “Synthesized” as used herein and understood in the art, refers to polynucleotides produced by purely chemical as opposed to enzymatic methods. “Wholly” synthesized DNA sequences are therefore produced entirely by chemical means, and “partially” synthesized DNAs embrace those wherein only portions of the resulting DNA were produced by chemical means. Genomic DNA of the invention comprises the protein-coding region for a polypeptide of the invention and is also intended to include allelic variants. Allelic variants. Allelic variants are understood to mean naturally-occurring base changes in the species population which may or may not result in an amino acid change of the DNA sequences herein.

“16S ribosomal RNA” is understood to mean an isolated small subunit RNA of any prokaryote whether isolated from ribosomes, made synthetically or prepared by transcription, “16S ribosomal RNA” can mean either the full length sequence or a fragment thereof.

As used herein, the term “contacting” means bringing together, either directly or indirectly, a compound into physical proximity to a polypeptide or polynucleotide of the invention. Additionally “contacting” may mean bringing a polypeptide of the invention into physical proximity with another polypeptide or polynucleotide (either another polypeptide or polynucleotide of the invention or a polypeptide or polynucleotide not so claimed) or bringing a polynucleotide of the invention into physical proximity with a polypeptide or polynucleotide (either a polypeptide or polynucleotide of the invention or a polypeptide or polynucleotide not so claimed).

As used herein, the term “polyribonucleotide protein complex” refers to a covalent or non-covalently associated molecular entity containing 16S ribosomal RNA and at least one small subunit ribosomal protein “Small subunit ribosomal protein” as used herein refers to ribosomal proteins present in the small (30S) ribosomal subunit of the ribosome of derived from any prokaryotic species. Small subunit ribosomal proteins include: S1, S2 S3, S4, S5, S6, S7, S8, S9, S11, S11, S12, S13, S14, S15, S16, S17, S18, S19, S20, and “Direct binding ribosomal polypeptide’ or “direct binding S-protein” or “direct binding ribosomal protein” or “direct binding protein” as used herein refers to a polypeptide derived from any prokaryotic species selected from the group consisting of S4, S7, S8, S17, S15 and S20 “Non-direct binding ribosomal polypeptide” or “non direct binding S-protein” or “non direct binding ribosomal protein” or “non-direct binding protein” as used herein refers to a polypeptide derived from any prokaryotic species selected from the group consisting of S1, S2 S3, S5, S6, S9, S10, S11, S12, S13, S14, S16, S18, S19, and S21. These proteins are also referred to as “secondary binding proteins”.

“Antibodies” as used herein includes monoclonal and polyclonal antibodies, chimeric, single chain, simianized antibodies and humanized antibodies, as well as Fab fragments, including the products of an Fab immunoglobulin expression library. The S20 ribosomal polypeptides of the invention or variants thereof, or cells expressing them can be used as an immunogen to produce antibodies immunospecific for such polypeptides.

Nucleic Acids of the Invention

A preferred DNA sequence of the invention encoding the Staphylococcus aureus S20 ribosomal polypeptide is set out in SEQ ID NO:1. The worker of skill in the art will readily appreciate that the preferred DNA of the invention comprises a double stranded molecule, for example the molecule having the sequence set forth in SEQ ID NO:1 along with the complementary molecule (the “non-coding strand” or “complement”) having a sequence deducible from the sequence of SEQ ID NO:1 according to Watson-Crick base pairing rules for DNA. Also preferred are other polynucleotides encoding the S20 ribosomal polypeptide of SEQ ID NO:2, which differ in sequence from the polynucleotide of SEQ ID NO:1 by virtue of the well-known known degeneracy of the universal genetic code. The determination of the nucleotide sequence is described in the following example.

### EXAMPLE 1

### Procedure for Obtaining Sequence Information of the S20 Gene Directly from the 2.8 Mb S. aureus Genome

The S. aureus S20 gene was sequenced using an ABI377 fluorescence-based sequencer (Perkin Elmer/Applied Biosystems Division, PE/ABD, Foster City, Calif.) and the ABI PRISM™ Ready Dye-Deoxy Terminator kit with Taq FS™ polymerase. Each ABI cycle sequencing reaction contained about 4 μg of Qiagen purified S. aureus genomic DNA, 100 ng of primer, and in a 2×standard reaction volume (40 μl total volume). Cycle-sequencing was performed using an initial denaturation at 98° C. for 1 min, followed by 100 cycles: 98° C. for 30 sec, annealing at 50° C. for 30 sec, and extension at 60° C. for 4 min. Temperature cycles and times were controlled by a Perkin-Elmer 9700 thermocycler. Extension products were purified using Centriflex™ gel filtration cartridges (Advanced Genetic Technologies Corp., Gaithersburg, Md.). Each reaction product was loaded by pipette onto the column, which was then centrifuged in a swinging bucket centrifuge (Sorvall model RT6000B table top centrifuge) at 1500×g for 4 min at room temperature. Column-purified samples were dried under vacuum for about 40 min and then dissolved in 1.5 μl of a DNA loading solution (83% deionized formamide, 8.3 mM EDTA, and 1.6 mg/ml Blue Dextran). The samples were then heated to 90° C. for three min and the complete sample was loaded into the gel sample well of the ABI377 sequencer. Sequence analysis was done by importing ABI377 files into the Sequencher program (Gene Codes, Ann Arbor, Mich.). Generally sequence reads of 600 bp were obtained. Sequence base call ambiguities were removed by obtained the complete sequence of each gene on both DNA strands.

Sequencing of the S. aureus S20 Gene.

Partial DNA sequences encoding a portion of S. aureus S20 ribosomal polypeptide have been described. Human Genome Sciences ID #V76479 and TIGR # TI:GSA_{—}604 The TIGR sequence matches the first 79 nucleotides of the sequence disclosed in this invention. The Human Genome Sciences, Inc. sequence contains 109 nucleotides which codes for the carboxy terminal 35 amino acid residues. The combination of the TIGR and HGS partial S20 ribosomal polypeptide gene sequences do not overlap as they contain a 63 nucleotide gap. The invention provides a complete sequence. The Bacillus subtilis ribosomal S20 polypeptide shares some identity with the S. aureus S20 ribosomal polypeptide; however the proteins differ by about 52% identity in their protein sequences.

The 187 bp GST in the TIGR database (TI:GSA 604) encodes about 26 amino acids of the S. aureus S20 ribosomal polypeptide gene starting with the Met codon.

This sequence, of unknown quality, was used to design three forward primers, SEQ ID NO:3 (5′AATATCAAATCTGCAATTAAACG) SEQ ID NO:4 (5′AAATTTTGATAAGATGAACTCAC) and SEQ ID NO:5 (5′TTTAGGAGGTGACAGAAATGGC). Only one of these primers generated any useful new sequence data, SEQ ID NO:3 primed a poor sequence read of about 400 bp. A second attempt using primer SEQ ID NO:3 produced a higher quality read that extended about 600 bp. Both reads were used to design three additional primers, forward primer SEQ ID NO:6. (5 ′ACGCAACATTTCACAAAAGAGTGC) and reverse primer SEQ ID NO:7 (5′-ATTGCACTCTTTTGTGAAATGTTGC) and SEQ ID NO:8 (5′-ATCTTTATAAAAAATAAAAGTTC). Excellent sequence reads of more than 500 bp. were obtained from primers SEQ ID NO:6 and SEQ ID NO:7 and a poor quality, but usable, read was obtained from primer SEQ ID NO:8. The combined four reads provided the complete double-stranded sequence of the S. aureus S20 ribosomal polypeptide gene region. Thus, the goal to obtain the complete accurate sequence of the S. aureus S20 ribosomal polypeptide gene directly from the genome was achieved. A total of 1.2 kb of sequence data was obtained within and around the S20 ribosomal polypeptide gene.

The invention further embraces species, which are homologs of the Staphyloccocus aureus S20 ribosomal polypeptide encoding DNA. Species homologs, would encompass nucleotide sequences which share at least 60%, at least 65%, at least 70%, at least 75%, at least 80%, at least 85%, at least 90%, at least 95%, at least 98%, at least 99% identity with Staphylococcus aureus polynucleotide of the invention

The polynucleotide sequence information provided by the invention makes possible large scale expression of the encoded polypeptide by techniques well known and routinely practiced in the art. Polynucleotides of the invention also permit identification and isolation of polynucleotides encoding related ribosomal proteins, such as allelic variants and species homologs, by well known techniques including Southern and/or Northern hybridization, and polymerase chain reaction (PCR).

The disclosure herein of a full length polynucleotide encoding an S20 ribosomal polypeptide makes readily available to the worker of ordinary skill in the art every possible fragment of the full length polynucleotide. The invention therefore provides fragments of the S20 ribosomal polypeptide encoding polynucleotides comprising at least 14-15, and preferably at least 18, 20, 25, 50, or 75 consecutive nucleotides of a polynucleotide encoding S20 ribosomal polypeptide. Preferably, fragment polynucleotides of the invention comprise sequences unique to the S20 ribosomal polypeptide encoding polynucleotide sequence and therefore hybridize under highly stringent or moderately stringent conditions only (i.e. “specifically”) to polynucleotides encoding S20 ribosomal polypeptide. Sequences unique to polynucleotides of the invention are recognizable through sequence comparison to other known polynucleotides, and can be identified through use of alignment programs routinely utilized in the art, e.g. those made available in public sequence databases. Such sequences are also recognizable from Southern hybridization analyses to determine the number of fragments of genomic DNA to which a polynucleotide will hybridize. Polynucleotides of the invention can be labelled in a manner that permits their detection, including radioactive, fluorescent, and enzymatic labelling.

Fragment polynucleotides are particularly useful as probes for detection of full length or other fragment S20 ribosomal polypeptide polynucleotides or for the expression of fragments of S20 ribosomal polypeptide. One or more fragment polynucleotides can be included in kits that are used to detect variations in a polynucleotide sequence encoding S20 ribosomal polypeptide.

The invention also embraces DNAs encoding S20 ribosomal polypeptide polypeptides which DNAs hybridize under moderately stringent or high stringency conditions to the non-coding strand, or complement, of the polynucleotide in SEQ ID NO:1

Exemplary highly stringent hybridization conditions are as follows: hybridization at 42° C. in a hybridization solution comprising 50% formamide, 1% SDS, 1M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel, et al. (Eds.), Protocols in Molecular Biology, John Wiley & Sons (1994), pp.6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

Host Cells and Vectors of the Invention

According to another aspect of the invention, host cells are provided, including prokaryotic and eukaryotic cells, comprising a polynucleotide of the invention (or vector of the invention) in a manner which permits expression of the encoded S20 ribosomal polypeptide. Polynucleotides of the invention may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, and mammalian cells systems. Suitable host cells for expression of S20 ribosomal polypeptides include prokaryotes, yeast, and higher eukaryotic cells. Suitable prokaryotic hosts to be used for the expression of human Staphylococcus aureus Ribosomal Protein Gene, S20 include bacteria of the genera Escherichia, Bacillus, and Salmonella, as well as members of the genera Pseudomonas, Streptomyces, and Staphylococcus.

The isolated nucleic acid molecules of the invention are preferably cloned into a vector designed for expression in prokaryotic cells, rather than into a vector designed for expression in eukaryotic cells. Prokaryotic cells are preferred for expression of genes obtained from prokaryotes because prokaryotic cells are more economical sources of protein production and because prokaryotic hosts grow to higher density and are typically grown in media which is less expensive than that used for the growth of eukaryotic hosts.

In the event a eukaryotic host were used the possibilities may include, but are not limited to, the following: insect cells, African green monkey kidney cells (COS cells), Chinese hamster ovary cells (CHO cells), human 293 cells, and murine 3T3 fibroblasts.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), and pQE vectors (Qiagen). A representative cloning and expression scheme is provided by the following example.

### EXAMPLE 2

### Isolation and Cloning of the S20 Coding Region

Two primers were designed for PCR. SEQ ID NO:9 (GTGTT ATCGATA ATGGCAAATATCAAATCTGCAATTAAACG)

This sequence includes an overhang (GTGTT), a Clal site, the start codon and the next 26 bases of the S20 ribosomal polypeptide gene and SEQ ID NO:10 (5′ GTGTTGGATCC TTA TTT ATT TGC AGT CAT TAA TTG TG). This sequence includes an overhang (GTGTT), a BamHl site, the stop codon and the next 23 bases of S20 S. aureus ribosoomal protein. Staphylococcus aureus genomic DNA was used as a template. The buffer (N808-0006) and Amplitaq® (N8080-0101) were purchased from Perkin Elmer Cetus. The 10 mM dNTP mix was obtained from Gibco BRL (Gaithersburg, Md.). The reaction mix was 5 μl of buffer, 1 μl of dNTP mix, 1 ng of each primer, 1 ng of genomic DNA and 0.5 μl (2.5 units) of amplitaq in a final volume of 50 μl. The program for PCR was 94° C. for 10 minutes and then 40 cycles of 94° C. for 1 minute, 57° C. for 30 seconds, and 72° C. for one minute. The final extension phase was at 72° C. for 3 minutes and the reactions were allowed to stay at 4° C. until they were removed from the thermocycler.

### Vector Construction and Expression

The PCR products were purified, digested with Cla1 and BamH1 and ligated to the expression vector pSR-Tac which contains Cla I and BamHI cloning sites. This vector contains a tac promoter, an AT rich synthetic ribosome binding site, two transcription terminators designated T1 and sib3 upstream of the tac promoter and downstream of the cloned gene, respectively, an ampicillin resistance gene derived from pBR322, and a ColE1 origin of replication. The Cla I restriction site is located immediately downstream of the ribosome binding site and the BamHI site is immediately upstream of the sib3 terminator. While this particular vector worked quite well it is expected that other vectors used in E. coli heterologous protein expression would be equally suitable.

After transformation into E. coli strain TopIO F′ laci^{q}, the colonies were screened by DNA mini prep and restriction digestion to find the desired constructs. The constructs were sequenced and transformed into E. coli strain K12s F′ laci^{q} for expression studies.

Cells harboring the construct pSRTac-S20 were grown in 50 ml LB with ampicillin at 37° C. The cultures were induced with 10⁻³ M IPTG during the midlog phase of growth and allowed to express for 3 hours. Then the cells were collected, sonicated and examined using gel electrophoresis.

Half a milliliter of the sonicated expression cultures were centrifuged at 10,000 rpm for 10 minutes. The supernatant was collected as the soluble fraction and the pellet (insoluble fraction) was suspended in 10 mM Tris-HCl pH 8.0. These samples were electrophoresed on 20% acrylamide with DATD crosslinker. The S20 protein was expressed at moderate levels and observed to be in the soluble fraction.

Polypeptides of the Invention

Overexpression in eukaryotic and prokaryotic hosts as described above facilitates the isolation of S20 polypeptides. The invention therefore includes isolated S20 polypeptides as set out in SEQ ID NO:2 and variants and conservative amino acid substitutions therein including labeled and tagged polypeptides.

The invention includes S20 polypeptides which are “labeled”. The term “labeled” is used herein to refer to the conjugating or covalent bonding of any suitable detectable group, including enzymes (e.g., horseradish peroxidase, beta glucuronidase, alkaline phosphatase, and beta-D-galactosidase), fluorescent labels (e.g., fluorescein, luciferase), and radiolabels (e.g., ¹⁴C, ¹²⁵I, ³H, ³²P, and ³⁵S) to the compound being labeled. Techniques for labeling various compounds, including proteins, peptides, and antibodies, are well known. See, e.g., Morrison, Methods in Enzymology 32b, 103 (1974); Syvanen et al., J. Biol. Chem. 284, 3762 (1973); Bolton and Hunter, Biochem. J. 133, 529 (1973). The termed labelled may also encompass a polypeptide which has covalently attached an amino acid tag as discussed below.

In addition, the S20 polypeptides of the invention may be indirectly labeled. This involves the covalent addition of a moiety to the polypeptide and subsequent coupling of the added moiety to a label or labeled compound which exhibits specific binding to the added moiety. Possibilities for indirect labeling include biotinylation of the peptide followed by binding to avidin coupled to one of the above label groups. Another example would be incubating a radiolabeled antibody specific for a histidine tag with a S20 polypeptide comprising a polyhistidine tag. The net effect is to bind the radioactive antibody to the polypeptide because of the considerable affinity of the antibody for the tag.

The invention also embraces variants (or analogs) of the S20 protein. In one example, insertion variants are provided wherein one or more amino acid residues supplement a S20 amino acid sequence. Insertions may be located at either or both termini of the protein, or may be positioned within internal regions of the S20 protein amino acid sequence. Insertional variants with additional residues at either or both termini can include for example, fusion proteins and proteins including amino acid tags or labels. Insertion variants include S20 polypeptides wherein one or more amino acid residues are added to a S20 acid sequence, or to a biologically active fragment thereof.

Insertional variants therefore can also include fusion proteins wherein the amino and/or carboxy termini of S20 is fused to another polypeptide. Various tag polypeptides and their respective antibodies are well known in the art. Examples include poly-histidine (poly-his) or poly-histidine-glycine (poly-his-gly) tags; the influenza HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397(1990)]. In addition, the S20 polypeptide can be tagged with enzymatic proteins such as peroxidase and alkaline phosphatase.

In another aspect, the invention provides deletion variants wherein one or more amino acid residues in a S20 polypeptide are removed. Deletions can be effected at one or both termini of the S20 polypeptide, or with removal of one or more residues within the S20 amino acid sequence. Deletion variants, therefore, include all fragments of the S20 polypeptide.

The invention also embraces polypeptide fragments of the sequence set out in SEQ ID NO: 2 wherein the fragments maintain biological (e.g., ligand binding or RNA binding and/or other biological activity) Fragments comprising at least 5, 10, 15, 20, 25, 30, 35, or 40 consecutive amino acids of SEQ ID NO: 2 are comprehended by the invention. Fragments of the invention having the desired biological properties can be prepared by any of the methods well known and routinely practiced in the art.

The present invention also includes include variants of the aforementioned polypetide, that is polypeptides that vary from the referents by conservative amino acid substitutions, whereby a residue is substituted by another with like characteristics. Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A (from WO 97/09433, page 10, published Mar. 13, 1997 (PCT/GB96/02197, filed Sep. 6, 1996), immediately below.

TABLE A
Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar- charged | D E |
| | K R |
| Aromatic | H F W Y |
| Other | N Q D E |

Alternatively, conservative amino acids can be grouped as described in Lehninger, [Biochemistry, Second Edition; Worth Publishers, Inc. NY:NY (1975), pp.71-77] as set out in Table B, immediately below

TABLE B
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still an another alternative, exemplary conservative substitutions are set out in Table C, immediately below.

TABLE C
Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

Generally it is anticipated that the S20 polypeptide will be found primarily intracellularly, the intracellular material can be extracted from the host cell using any standard technique known to the skilled artisan. For example, the host cells can be lysed to release the contents of the periplasm/cytoplasm by French press, homogenization, and/or sonication followed by centrifugation. The S20 polypeptide is found primarily in the supernatant after centrifugation of the cell homogenate, and the S20 polypeptide can be isolated by way of non-limiting example by any of the methods below. In those situations where it is preferable to partially or completely isolate the S20 polypeptide, purification can be accomplished using standard methods well known to the skilled artisan. Such methods include, without limitation, separation by electrophoresis followed by electroelution, various types of chromatography (immunoaffinity, molecular sieve, and/or ion exchange), and/or high pressure liquid chromatography. In some cases, it may be preferable to use more than one of these methods for complete purification.

Purification of S20 polypeptide can be accomplished using a variety of techniques. If the polypeptide has been synthesized such that it contains a tag such as Hexahistidine (S20/hexaHis) or other small peptide such as FLAG (Eastman Kodak Co., New Haven, Conn.) or myc (Invitrogen,Carlsbad, Calif.) at either its carboxyl or amino terminus, it may essentially be purified in a one-step process by passing the solution through an affinity column where the column matrix has a high affinity for the tag or for the polypeptide directly (i.e., a monoclonal antibody specifically recognizing S20). For example, polyhistidine binds with great affinity and specificity to nickel, thus an affinity column of nickel (such as the Qiagen Registered ™ nickel columns) can be used for purification of S20/polyHis. (See for example, Ausubel et al., eds., Current Protocols in Molecular Biology, Section 10.11.8, John Wiley & Sons, New York [1993]).

Even if the S20 polypeptide is prepared without a label or tag to facilitate purification. The S20 of the invention may be purified by immunoaffinity chromatography. To accomplish this, antibodies specific for the S20 polypeptide must be prepared by means well known in the art. Antibodies generated against the S20 polypeptides of the invention can be obtained by administering the polypeptides or epitope-bearing fragments, analogues or cells to an animal, preferably a nonhuman, using routine protocols. For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in Monoclonal Antibodies and Cancer Therapy, Alan R. Liss, Inc. (1985).

Where the S20 polypeptide is prepared without a tag attached, and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity. A representative purification scheme is detailed below.

### EXAMPLE 3

### Large Scale Purification of S20 Protein

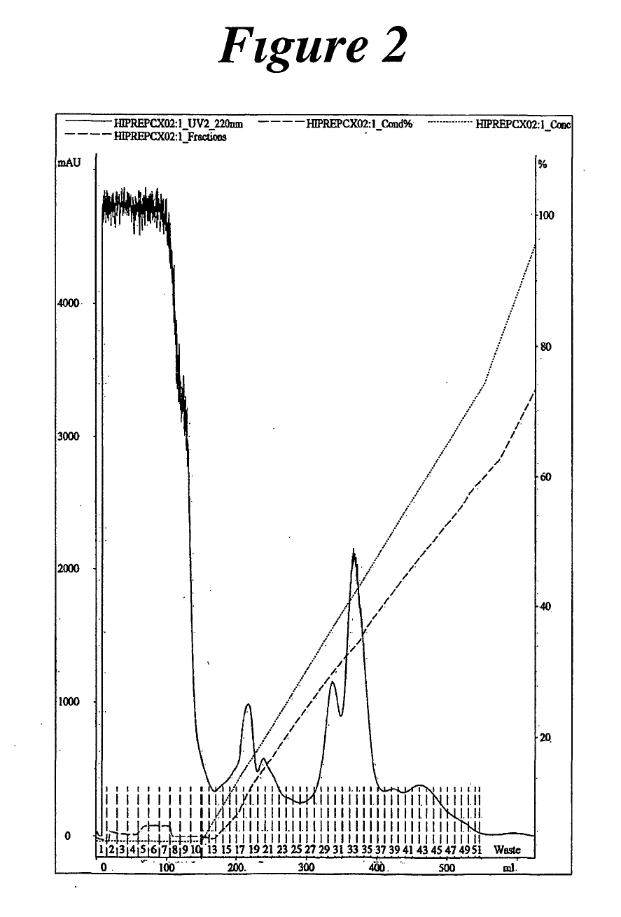

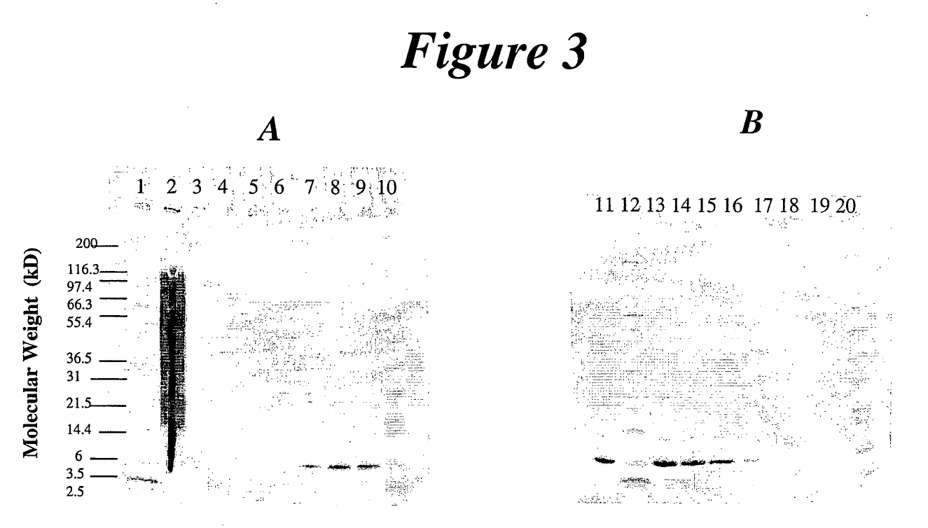

S20-expressing E. coli cell paste resulting from 6 liters of fermentation was resuspended in ˜70 mL Tris buffer pH 7.4 containing 1 mM MgCl₂ and 1 mM DTT. One Completee EDTA-free protease inhibitor pellet (Boehringer Mannheim, Indianapolis, Ind.) was added to the suspended cells. The cells were lysed by passage three times through a French Press @ 10,000 PSI. A soluble fraction was prepared from the cellular lysate by ultracentrifugation @ 100,000×g for 60 minutes @ 4° C. The soluble fraction was injected onto a HiPrep SP_{XL} 16/10 cation exchange column which had been equilibrated in 50 mM Tris buffer pH 7.4, 1 mM MgCl₂, and 1 mM DTT. The column flow rate was 4 mL/min. The column was washed with buffer until the Abs₂₈₀ of the column eluate was less then 0.01. Material was eluted off of the HiPrep SP_{XL} column with a linear gradient of 0-700 mM NaCl in column buffer over 20 column volumes. The column profile is shown in FIG. 2. Fractions were collected and analyzed by SDS-PAGE using 4-12% Bis-Tris NuPagee gels (Novex, San Deigo, Calif.) employing a MES buffer system. The gel is shown in FIG. 3. The gel legend is shown below.

Key to S20 Gel

| Lane | Sample | Lane | Sample |
|---|---|---|---|
| 1 | MW Standards | 11 | Fraction 32 |
| 2 | Crude Lysate | 12 | MW Standards |
| 3 | Fraction 25 | 13 | Fraction 33 |
| 4 | Fraction 26 | 14 | Fraction 34 |
| 5 | Fraction 27 | 15 | Fraction 35 |
| 6 | Fraction 28 | 16 | Fraction 36 |
| 7 | Fraction 29 | 17 | Fraction 37 |
| 8 | Fraction 30 | 18 | Fraction 38 |
| 9 | Fraction 31 | 19 | Fraction 39 |
| 10 | MW Standards | 20 | MW Standards |

S20-containing fractions were further analyzed by liquid chromatography electrospray mass spectrometry (LC/MS-ESI) performed on a Finnigan LC/Q instrument. The results of the LC/MS-ESI analysis yielded an average mass of 8064 amu which would correspond to a des⁹ form of S. aureus ribosomal protein S20. The calculated average mass of the intact S20 is calculated to be 9021.46. The calculated average mass of the des⁹ form of S20 is 8064.25. The sequence of S. aureus S20 is shown below The des⁹ form of the protein is highlighted in bold type

MANIKSAIKRVKTTEKAEARNISQKSAMRTAVKNAKTAVSNNADNKNELVSLAVKLVD AQSNLIHSNKADRIKSQLMTANK

In addition to preparing and purifying S20 polypeptide using recombinant DNA techniques, the S20 polypeptides, fragments, and/or derivatives thereof may be prepared by chemical synthesis methods (such as solid phase peptide synthesis) using techniques known in the art such as those set forth by Merrifield et al., (J. Am. Chem. Soc., 85:2149 [1963]), Houghten et al. (Proc Natl Acad. Sci. USA, 82:5132 [1985]), and Stewart and Young (Solid Phase Peptide Synthesis, Pierce Chemical Co., Rockford, Ill. [1984]). Such polypeptides may be synthesized with or without a methionine on the amino terminus. Chemically synthesized S20 polypeptides or fragments may be oxidized using methods set forth in these references to form disulfide bridges. The S20 polypeptides or fragments are expected to have biological activity comparable to S20 polypeptides produced recombinantly or purified from natural sources, and thus may be used interchangeably with recombinant or natural S20 polypeptide.

Ribosomal Assembly Assays 70S ribosome particles in E. coli consist of 31 core ribosomal “L” proteins and two rRNAs (5S and 23S) in the 50S subunit and 21 “S” proteins and a single 16S rRNA in the 30S subunit. These particles constitute the basic machinery for bacterial protein translation. It is postulated that the Staphylococcus aureus ribosome is assembled in fashion to ribosomes in E. coli. The present invention provides several methods to study the S. aureus 30S subunit assembly and methods to screen for inhibitors of the assembly process.

Assembly of the 30S ribosomal subunit is an ordered process both in vivo and in vitro. Nomura, M. and Held, W. A. (1974), Noller and Nomura (1987). It is now well known that the 21 proteins which comprise the the E. coli 30S subunit assemble onto the the 16S rRNA in an ordered fashion in vitro. Id. These proteins have been defined as primary or secondary binders, according to whether they bind to the 16S RNA independently of other proteins or not. Proteins that bind directly to 16S rRNA include S4, S7, S8, S15, S17 and S20. Secondary binding proteins include S3, S5, S9, S10, S12, S14, S16 and S19.

Producing and purifying the S. aureus ribosomal “S” proteins which are most critical for the formation of functional 30S subunits including those that bind directly to 16S rRNA (i.e., S4, S7, S8, S15, S17 and S20) “direct binding S-proteins” and critical proteins that integrate themselves into the ribosome by reliance on protein-protein and/or protein-RNA interactions (non-direct binding S-proteins)(S3, S5, S9, S10, S12, S14, S16 and S19) provides myriad choices in designing methods for testing inhibitors of ribosomal assembly.

16S RNA Binding Assay for Ribosomal Protein S20

Because S20 is a direct binding S protein it makes possible an assay in which S20 binding to 16S RNA may be measured directly. Such an assay involves the incubation of S20 polypeptide with 16S RNA, separation of bound from unbound S20 and measurement of that fraction of the S20 that remains bound to the RNA. By way of non-limiting example one can envision numerous ways in which the presence of unbound or bound S20 could be detected. The S20 might be radiolabeled in any of a number of means including but not limited to, labeling in vitro by chemical or enzymatic means or vivo by metabolically labeling cells expressing S20.

As discussed above commonly used radioactive isotopes used for the radiolabeling of peptides and proteins and nucleic acids include but are not limited to ³H, ¹⁴C, ³⁵S, 1251 and ³²P. In addition, of course, if the S20 polypeptide or is tagged with an amino acid tag, as described above, the tag and the covalently attached S20 protein can be detected by means well known in the art. In addition, the S20 polypeptide or a polynucleotide can be tagged with enzymatic proteins such as peroxidase and alkaline phosphatase, and fluorescent labels (U.S. Pat. No. 3,940,475) which are capable of being monitored for change in fluorescence intensity, wavelength shift, or fluorescence polarization (FP) or fluorescent resonance energy transfer (FRET). Another method of labeling polypeptides and nucleic acids includes biotinylation of the peptide of the peptide or nucleic acid followed by binding to avidin coupled to one of the above label groups or a solid support. In addition of course, such an assay is amenable to being performed with the 16S RNA (or a fragment thereof) being labeled with a radiolabel, a tag, or indirectly with a molecule such as biotin. The assay may be performed entirely in solution phase or it may be performed with either the 16S RNA or the 20S polypeptide immobilized. A common means of immobilization is to attach biotin to the molecule of interest and immobilize it by contacting with a solid support to which avidin is bound. By way of non-limiting example, an assay in which the S20 polypeptide is immobilized on a solid support and is used to bind radiolabeled 16S RNA and an assay in which all components are free in solution are described below.

### EXAMPLE 4

### 16S RNA-S20 Binding Assay

Because S20 is known to bind directly to 16S rRNA isolated S20 protein is an important reagent for developing a protein:RNA binding assay. The reagents for such a screen include S20 protein and labeled 16S RNA or a fragment of 16S RNA capable of binding the S20 polypeptide. Depending on the format of the assay, the S20 polypeptide or the 16S RNA may be labeled by means of radiolabeling or with tags which make the RNA or polypeptide amenable to immobilization to a solid support.

Preparation of Starting Materials

Cloning of 16S Ribosomal RNA

The complete 16S-rRNA gene was identified in the HGS data base on contig 168268 by homology to the B. subtilis sequence. Five prime sequence of 5′TTTATGGAGAGTTTGATCCTGGC-3′ and the 3′ sequence of 5′GCGGCTGGATCACCTCCTTTCT-3′is used to amplify the entire 16S-rRNA gene from S. aureus (Oligo Etc; Wilsonville, Oreg.). The amplified gene is cloned into pT7Blue using Novagen's (Madison, Wis.) Perfectly Blunt Cloning Kit. DNA template is created by PCR using a primer that had the T7 promoter on the 5′ end sequence of the 16S-rRNA gene (5′-TAATACGACTCACTATAGTTTTATGGAGAGTTTGATCCTGGC-3′). The length of the amplified 16S-rRNA fragment can be altered by the selection of the 3′ primer. Whole 16S-rRNA as well as shorter segments could be used for screening of S20-16S-rRNA antagQnists. The crystal structure has been solved for the 30S subunit (Brian T. Wimberly, et al Structure of the 30S ribosomal subunit. Nature. vol 407; p327-338, 2000). Helical pieces, H8, H9, H11, and H44 create a pocket for the S20 protein to bind. These smaller helical pieces can be used for screen of S20 antagonist. Fragmented segments can be generated with the same T7 promoter as the whole 16S-rRNA was created and can also be labeled.

| Helical RNAs | 5′ | 3′ |
|---|---|---|
| H-44 | CACCACGAGAGTTTGTAAC | CACCCCAATCATTTGTCCCAC |
| Nucleotide 1419-1502 | (SEQ ID NO: 59) | (SEQ ID NO: 60) |
| SEQ ID NO: 21 | | |
| H-7, 8, 9, 10, & 11 | CACGTGGATAACCTACCTA | GTGGCCGATCACCCTCTCAGG |
| Nucleotide 120-322 | (SEQ ID NO: 61) | (SEQ ID NO: 62) |
| SEQ ID NO: 21 | | |

³H-UTP or ³⁵S-ATP can be used to label the RNA if labeled RNA is desired. Resulting RNAs are characterized by electrophoresis on acrylamide-urea gels, and RNA concentrations are determined by UV spectroscopy using A₂₆₀ unit = 40 ug/ml. The entire S16 ribosomal RNA gene sequence has been reported (Genbank Accession # X68417 also U.S. Pat. No. 5,843,669 Sequence # 160). The sequence of the gene is included in this document as SEQ ID NO: 21

Biotinylation of 520

Purified S20 is biotinylated with the Pierce EZ-link Sulfo-NHS-LC-Biotinylation Kit (Pierce, Rockford, Ill.). Briefly, 40 μl of S20 (about 6.0 mg/ml), 64 μl of Sulfo-NHS-LC-Biotin (10 mg/ml), and 598 μl of kit PBS buffer is allowed to react on ice for 2 hours. Excess biotin is removed by column desalting, dialysis or both. Desalting is performed by adding the product to a 10 ml desalting column that had been equilibrated with 30 ml of PBS buffer. The one milliliter sample is allowed to permeate the gel and 1 ml fractions is collected. Fractions are monitored by the Bio Rad Protein Assay (Bio Rad, Hercules, Calif.). Dialysis is performed using a Pierce Slide-A-Lyzer 10K cassette (Pierce, Rockford, Ill.), under constant stirring for 16 hours at 4° C. against 2 liters of 30 mM Phosphate buffer (pH 7.0), 400 mM NaCl.

Multiscreen Assay and Scintillation Proximity Assay (SPA)

The binding assay reported by Vartikar (1989) is modified as follows: S20 was diluted into TK buffer (350 mM KCl, 10 mM β-mecaptoethanol, 30 mM Tris [pH 7.6]) and incubated at 37° C. for 30 minutes. Labelled RNA is renatured in buffer (350 mM KCl, 20 mM MgSO₄, 10 mM 13-mecaptoethanol, 30 mM Tris [pH 7.6]) at 40° C. for 20 minutes. After renaturation, the S20 (30 μl) and 16S-rRNA (20 μl) is incubated at 0 room temperature for 10 minutes. A Multiscreen HA opaque 96 well filtration plate (Millipore; Bedford, Mass.) is first prewetted with 100 μl of Dulbecco's PBS for 10 minutes and vacuumed to remove excess fluid. The S20-16S-rRNA complex is transferred to the Multiscreen plate, incubated for 5 minutes, vacuumed, air dried for 1 hour, and counted with 40 μl of scintillation cocktail on a Topcount™ Microplate Scintillation Counter. The SPA assay is run almost identical to the Multiscreen assay except that it utilized biotinylated S20 and strepavidin coated SPA beads (Amersham) in the final reaction. As before the S20 and 16S-rRNA is allowed to react for 10 minutes. Fifty μl of SPA beads (20 mg/ml) is added to the 50 μl of S20: 16S-rRNA complex in a Dynatech Microlite plate and counted in a Topcountr Microplate Scintillation Counter. Inhibition studies are conducted with 16S/23S-rRNA and MS2-mRNA purchased from Roche Molecular Biochemicals, Indianapolis, Ind. To identify potential inhibitors of the 16S RNA-20S complex the assay is run in the presence and absence of potential inhibitors and the effect on binding is assessed.

Simultaneous Assay of S4, S7, S8, S15, S17 and S20 Binding to 16S RNA:

While the discussion above, illustrates an assay useful for the identification of inhibitors which directly disrupt the interaction between the S20 polypeptide and the 16S ribosomal RNA. It is recognized that the binding of the S20 polypeptide may, in part, be dependent on the interaction of other direct binding S-proteins binding in concert to the 16S ribosomal RNA. Such dependence may be the result of alterations in the conformation of the 16S ribosomal RNA or

In another embodiment, all the direct binding S-proteins can be incubated with 16S RNA and the presence of bound or unbound S20 polypeptide determined. Indeed, the identity of all of the bound or unbound proteins can be determined. The identity of a bound or unbound S protein can be determined, for instance by a suitable mass spectrometry technique, such as matrix-assisted laser desorption/ionization combined with time-of-flight mass analysis (MALDI-TOF MS) or electrospray ionization mass spectrometry (ESI MS). See Jensen et al., 1977, Protein Analysis By Mass Spectrometry, In Creighton (ed.), Protein Structure, A Practical Approach (Oxford University Press), Oxford, pp. 29-57; Patterson & Aebersold, 1995, Electrophoresis 16: 1791-1814; Figeys et al., 1996, Analyt. Chem. 68: 1822-1828 (each of which is incorporated herein by reference in its entirety). Preferably, a separation technique such as HPLC or capillary electrophoresis is directly or indirectly coupled to the mass spectrometer. See Ducret et al., 1996, Electrophoresis 17: 866-876; Gevaert et al., 1996, Electrophoresis 17: 918-924; Clauser et al., 1995, Proc. Natl. Acad. Sci. USA 92: 5072-5076 (each of which is incorporated herein by reference in its entirety).

### EXAMPLE 5

### Assay of S20 with Other Direct Binding Proteins

This assay is used to test for direct RNA:protein assembly. The starting material proteins are preferably prepared by recombinant means and over-expression in a suitable host essentially as described in Examples 1, 2 and 3 for S20 with obvious modifications to reflect the differing sequences of the proteins involved. The nucleotide sequences of cDNA's encoding S. aureus direct binding ribosomal proteins S4, S7, S8, S15 and S17 are presented in SEQ ID NOS:11, 13, 15, 17 and 19 respectively. Sequences encoding S4, S7, S8, S15, and S17 can be isolated by means of the polymerase chain reaction. Primers are selected such that entire coding region is isolated. The complete amino acid sequences of S4, S7, S8, S15, and S17 polypeptides are presented in SEQ ID NOS:12, 14, 16, 18 and 20. Sequences encoding S4, S7, S8, S15, and S17 can be isolated by means of probing a genomic Staphylococcus aureus library with probes designed from SEQ ID NOS:11, 13, 15, 17 and 19 as well. The polymerase chain reaction would be a preferred method because it generally allows the isolation of a complete coding sequence in one experiment.

Methods for preparing and using probes and primers are described, for example, in Sambrook et al., Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1987 (with periodic updates); and Innis et al., PCR Protocols: A Guide to Methods and Applications, Academic Press: San Diego, 1990.

Primers are selected to have low self- or cross-complementarity, particularly at the 3′ ends of the sequence. Long homopolymer tracts and high GC content are avoided to reduce spurious primer extension. Primers are typically about 20 residues in length, but this length can be modified as well-known in the art, in view of the particular sequence to be amplified. Computer programs are available to aid in these aspects of the design. One widely used computer program for designing PCR primers is (OLIGO 4.0 by National Biosciences, Inc., 3650 Annapolis Lane, Plymouth, Mich.). Another is Primer (Version 0.5,(c) 1991, Whitehead Institute for Biomedical Research, Cambridge, Mass.).

Isolated 16S RNA is Prepared as Described in Example 4.

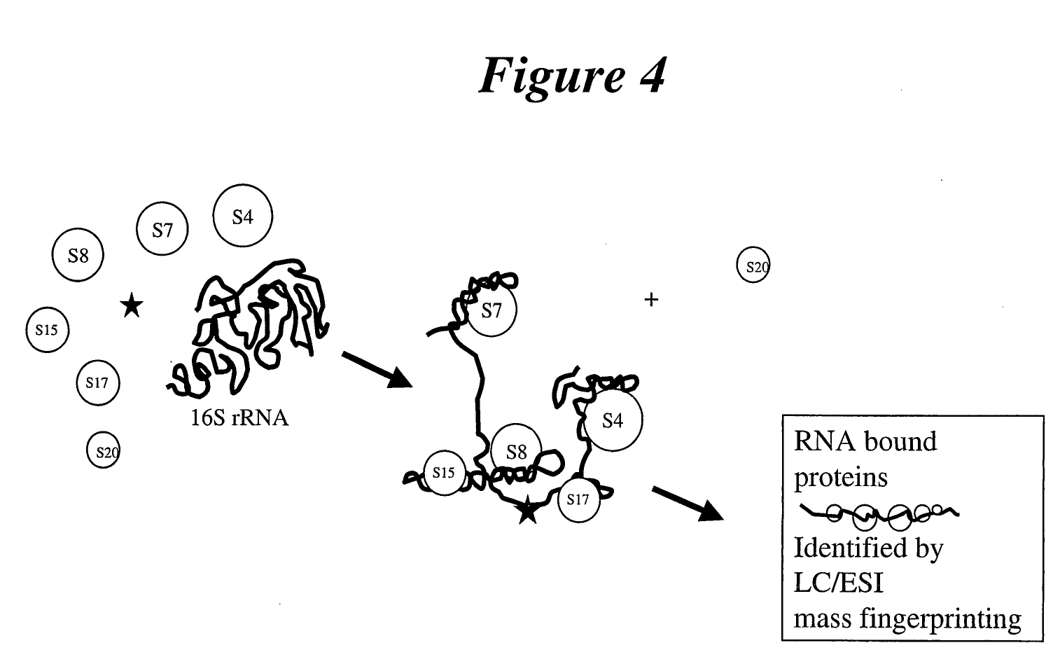

In this assay all six of the S-proteins that bind directly to 16S RNA are added together with test compound. Unbound S-proteins are then removed by size-separation or filtration. Automated LC/ESI ion-trap or MALDI-to-MS is then used to determine if a particular S-protein is inhibited in its binding to 16S RNA. Mass spectrometry is an ideal detection tool since all of the S-protein average masses are known and unique. An example illustrates how specific inhibition of S20 protein binding to RNA is detected. The concept is illustrated in FIG. 4.

RNA:protein assembly is assayed in 80 mM K⁺-HEPES, pH 7.6, 20 mM MgCl₂, 330 mM NaCl at 42° C. The procedure is based on the conditions of Culver and Noller (RNA, 1999, 5: 832-843) except that 0.01% Nikkol detergent is removed because it significantly complicates the LC/MS analysis. Primary ribosomal binding proteins S4, S7, S8, S15, S17, and S20 are dialyzed overnight against 80 mM K⁺-HEPES, pH 7.6, 20 mM MgCl₂, 1 M NaCl. In the reconstitution, 200 μmol in vitro transcribed 16S RNA is incubated at 42° C. for 15 minutes. Then, 800 μmol S7, S8, S15, S17, and S4 each are added to the RNA, followed by 400 μmol S20. The NaCl concentration is then adjusted to 330 mM by adding 80 mM K⁺-HEPES, pH 7.6, 20 mM MgCl₂. The mixture is then incubated at 42° C. for 20 more minutes. The protein:RNA complex is then separated from the free proteins by spinning in a YM100 Microcon at 500×g for 20 minutes. The RNA is precipitated from the retentate by adding 2 volumes of acetic acid and incubating on ice for 45 minutes. Proteins from both the flow-through and retentate are analyzed by LC/ESI ion trap mass spectrometry. The proteins are first separated on a C4 reversed phase column (Vydac) using a gradient from 98% of 0.1% TFA, 2% of 90% acetonitrile/0.1% TFA to 100% of 90% acetonitrile/0.1% TFA. The intact mass of each protein are observed by electrospray mass spectrometry as it eluted from the column.

We have also been able to identify S20 in a mixture of primary ribosomal binding proteins by MALDI-TOF mass spectrometry. The mixture of proteins is passed over a C18 zip-tip (Millipore) to remove salts, eluting in 80% acetonitrile/0.1% TFA. A saturated solution of sinapinic acid is prepared in 30% acetonitrile/0.1% TFA. One microliter of the protein solution is mixed with ten microliters of the matrix solution, and 0.5 μL is spotted onto the stainless steel MALDI target. MALDI-TOF data were collected in linear mode from 6000-25000 Da, and the intact mass for S20 is observed.

Of course, purified direct binding proteins make possible assays to access the association of any or all direct binding proteins with 16S RNA. The invention of course, includes methods for testing for inhibitors of ribosomal assembly in which the incorporation of any direct binding protein into the polyribonucleotide protein complex is accessed.

### EXAMPLE 6

### Scintillation Proxinmity Assay (SPA) Assay of S20 with Other Direct Binding Proteins

As in the previous example all S4, S7, S8, S15 and S17 are incubated together with 16S RNA followed by S20 ribosomal polypeptide in the presence and absence of a test compound. Starting materials are prepared roughly as described in previous examples. In this example the 16S ribosomal RNA is end labeled with biotin and the S20 ribosomal polypeptide is radioactively labeled.

Primary ribosomal binding proteins S4, S7, S8, S15, S17, and S20 are dialyzed overnight against 80 mM K⁺-HEPES, pH 7.6, 20 mM MgCl₂, 1 M NaCl. In the reconstitution, 200 pmol in vitro transcribed 16S RNA is incubated at 42° C. for 15 minutes. Then, 800 pmol S7, S8, S15, S17, and S4 each are added to the RNA, followed by 400 pmol S20. The NaCl concentration is then adjusted to 330 nm by adding 80 mM K⁺-HEPES, pH 7.6, 20 mM MgCl₂. Fifty μl strepavidin coated SPA beads (20 mg/ml) is added to the 50 t of of the reaction mixture in a Dynatech Microlite plate and counted in a Topcount™ Microplate Scintillation Counter. To identify potential inhibitors of S20 incorporation into the polyribonucleotide-protein complex, the assay is run in the presence and absence of potential inhibitors and the effect on binding is assessed.

Protein-protein Interaction Assembly Screen

The isolated S20 polypeptide of the invention also makes possible an assay through which one may detect all possible protein-protein disruptions in the 30S assembly process. This is important since published assembly maps are not based on the myriad of possible protein-protein interactions that may occur. In practice these maps are based on limited S-protein combinations that were tested in vitro. This assay makes use of the fact that the assembly of ribosomes in general and the 30S subunit in particular, is an ordered process and makes use of all 21 small subunit ribosomal proteins or a limited subset of those proteins. The S3 ribosomal protein is known to integrate itself last or very late in the ribosomal assembly process. Its efficient integration is known to be dependent upon the proper integration of the direct binding ribosomal proteins as well non-direct binding proteins. Proper partial assembly is monitored by measuring the incorporation of S3 ribosomal polypeptide into the partially or fully assembled ribosome. In the alternative, improper or disrupted assembly can be assayed by exclusion of S3 ribosomal polypeptide from the ribosome The S3 ribosomal protein may be labeled as discussed hereinbefore for ease of detection. The 16S ribosomal RNA or a direct binding ribosomal peptide may immobilized or the entire assay may be performed with all components in solution phase. The starting materials for the assays are preferably prepared by recombinant means. The DNA sequences encoding all 21 30S subunit proteins are provided in the sequence listings as well as the amino acids sequences encoded by each. The invention provides ribosomal assembly assays utilizing all 21 small subunit ribosomal proteins as well as a select subset of proteins readily apparent to one skilled in the art. Sequences encoding each protein can be isolated by means of the polymerase chain reaction. Primers are selected as discussed previously. Primers are selected as discussed previously. Primers are selected such that entire coding region is isolated. Methods for preparing and using probes and primers are discussed above.

Exemplary forward and reverse primers suitable for amplification of S4, S6, and S18 are described listed here by way of example. One skilled in the art would recognize that other primers may be equally suitable.

| | | |
|---|---|---|
| S4 Forward | 5′-TATATTATCGATAATGGCTCGATTCAGAGGT-3′ | (SEQ ID NO:53) |
| S4 Reverse | 5′-TATAGGATCCTTAACGGATTAATTGTTCGTTAATTT-3′ | (SEQ ID NO:54) |
| S18 Forward | 5′-TATATTATCGATAATGGCAGGTGGACCAAGAAG-3′ | (SEQ ID NO:55) |
| S18 Reverse | 5′TATAGGATCCTTATTGTTCTTCTTTAACAT-3′ | (SEQ ID NO:56) |
| S6 Forward | 5′-TATATTATCGATAATGAAGAAACATATGAAGTTAT-3′ | (SEQ ID NO:57) |
| S6 Reverse | 5′-TATAGGATCCTTACTTGTCTTCGTCTTCAC-3′ | (SEQ ID NO:58) |

The following is provided by way of non-limiting example.

### EXAMPLE 7

### Partial Ribosomal Assembly Assay

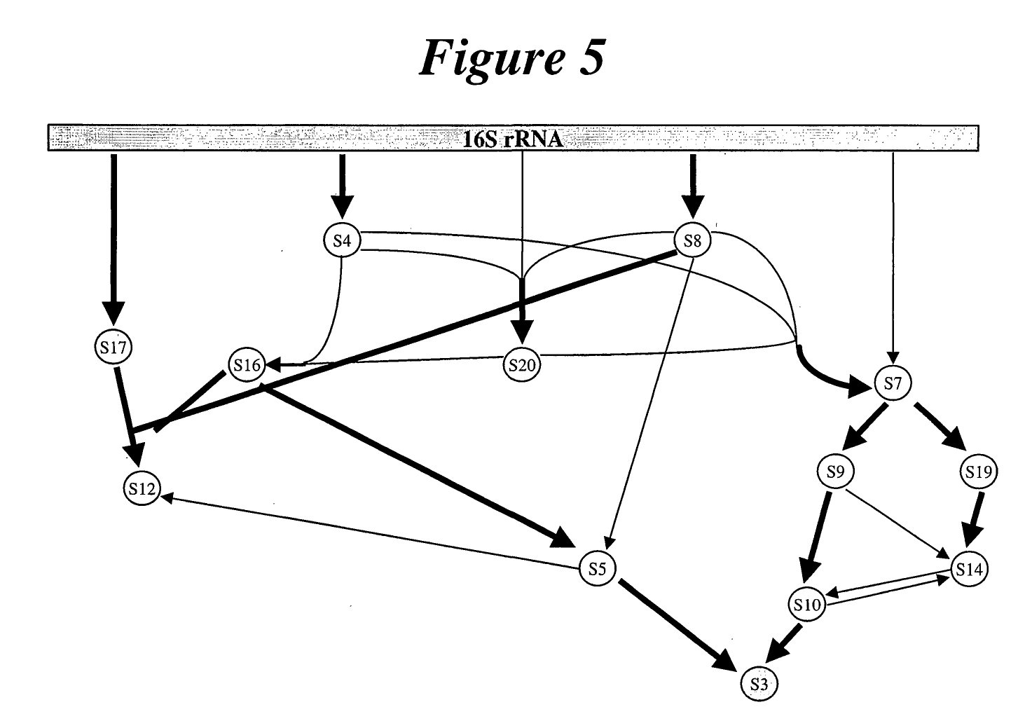

In this assay format several S-proteins are allowed to interact with 16S RNA in the presence of a test compound (FIG. 5). The assay makes use of all of the direct binding ribosomal proteins except S15 (S4, S7, S8, S17 and S20) and a select group of S. aureus ribosomal proteins which integrate themselves into the ribosome by reliance on protein-protein or protein-RNA interactions (S3, S5, S9, S10, S12, S14, S16 and S19)

The starting material proteins are prepared by recombinant means and over-expression in a suitable host essentially as described in Examples 1, 2 and 3 for the S20 polypeptide of the invention with obvious modifications to reflect the differing sequences of the proteins involved. The nucleotide sequences of cDNA's encoding S. aureus direct binding ribosomal proteins S4, S7, S8, and S17 are presented in SEQ ID NOS:11, 13, 15, and 19 respectively. The production of the isolated S20 polypeptide of the invention is described hereinbefore.

The nucleotide sequences of cDNA's encoding S. aureus ribosomal proteins which integrate themselves into the ribosome by reliance on protein-protein or protein-RNA interactions (non-direct binding ribosomal proteins) S3, S5, S9, S10, S12, S14, S16 and S19 are presented in SEQ ID NOS: 26, 28, 32, 34, 38, 42, 44, and 48 respectively. Nucleotide sequences encoding S. aureus. S3, S4, S5, S7, S8, S9, S10, S12, S14, S16 S17 and S19 can be isolated by means of the polymerase chain reaction. Primers are selected such that the entire amino acid coding region is isolated. The complete amino acid sequences of S. aureus S3, S4, S5, S7, S8, S9, S10, S12, S14, S16 S17 and S19 polypeptides are presented in SEQ ID NOS:27, 12, 29, 14, 16, 33, 35, 39, 43, 45, 20 and 49. Sequences encoding S3, S4, S5, S7, S8, S9, S10, S12, S14, S16 S17 and S19 can be isolated by means of probing a genomic Staphylococcus aureus library with probes designed from SEQ ID NOS:12, 28, 13, 15, 32, 34, 38, 42, 44, 19, and 48 as well. The polymerase chain reaction would be a preferred method because it generally allows the isolation of a complete coding sequence in one experiment. The S3 protein is labeled, preferably radiolabeled.

RNA:protein assembly is assayed in 80 mM K⁺-HEPES, pH 7.6, 20 mM MgCl₂, 330 mM NaCl at 42° C. The procedure is based on the conditions of Culver and Noller (RNA, 1999, 5: 832-843) except that 0.01% Nikkol detergent is removed because it significantly complicats the LC/MS analysis. Ribosomal proteins S3, S4, S5, S7, S8, S9, S10, S12, S14, S16, S17, S19 and S20 are dialyzed overnight against 80 mM K⁺-HEPES, pH 7.6, 20 mM MgCl₂, 1 M NaCl. In the reconstitution, 200 pmol in vitro transcribed 16S RNA is incubated at 42° C. for 15 minutes. Then, 800 pmol ribosomal proteins S4, S7, S8, S17, and S20 added to the RNA, followed by ribosomal proteins, S5, S9, S10, S12, S14, S16 and S19. The NaCl concentration is then adjusted to 330 mM by adding 80 mM K⁺-HEPES, pH 7.6, 20 mM MgCl₂. The mixture is then incubated at 42° C. for 20 more minutes. 800 pmol labeled ribosomal protein S3 is then added.

Unbound S-proteins are removed by size-separation or filtration. If the labelled S3 protein is present in the RNA:multiprotein complex then the compound does not inhibit any specific protein-protein interactions during the assembly process. If the compound prevents the incorporation of labelled S3 protein then the assay reveals that the test compound inhibits a protein-protein interaction.

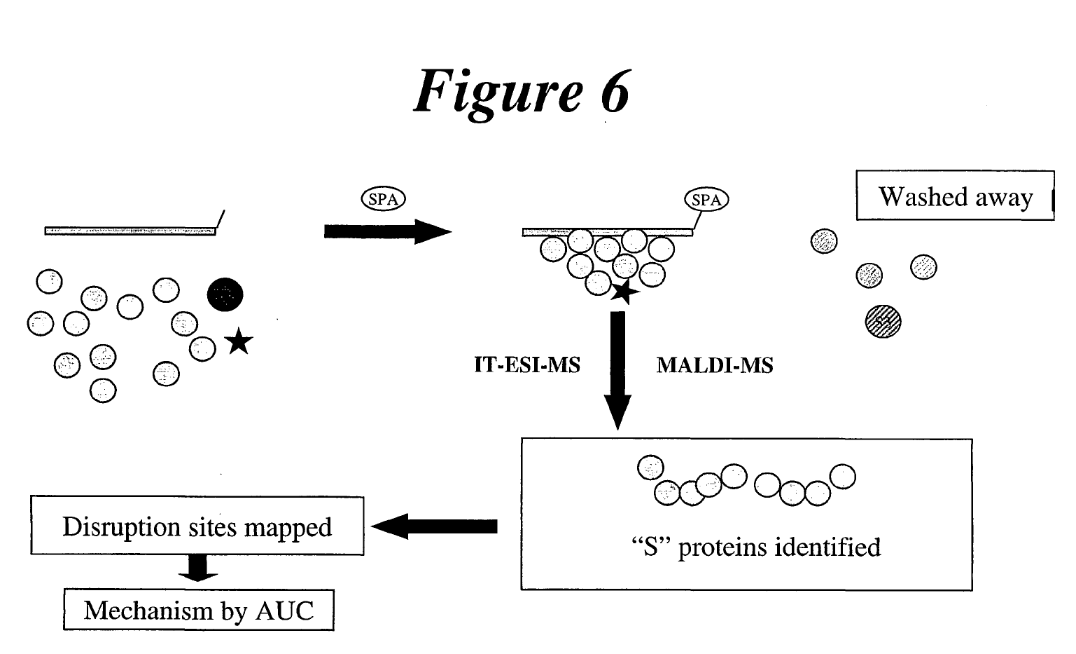

The partially assembled RNA:multiprotein complex is then analyzed by LC/ion-trap electrospray analysis to determine the S-protein components in the partially assembled complex. Alternatively MALDI-of-MS can be used. Knowing the identity of S-proteins in the partially assembled complex and published knowledge of how the 30S subunit is assembled in vitro (Noller and Nomura (1987) the protein-protein interaction that is disrupted by the test compound may be determined. The exact protein-protein interaction that is disrupted can be determined using selective combinations of S-proteins added to 16S RNA and compound. As stated above, this is an important confirmation process since published in vitro assembly maps are based on a limited data set. Assembly disruption by the test compound can be independently verified by analytical ultracentrifugation analysis (FIG. 6). In this process the partially assembled 30S complex is differentiated from intact complex by displaying a lower rate of sedimentation in a given centrifugal field (i.e., as measured by a lower sedimentation constant, expressed in Svedberg units or S). The contents of sedimentation clusters can be verified by mass spectrometry.

It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference.

What is claimed is:

1. An isolated nucleic acid comprising a nucleotide sequence that encodes an amino acid sequence having at least 85% identity with SEQ ID NO:2

2. An isolated nucleic acid comprising the nucleotide sequence having least 85% identity with SEQ ID NO:1

3. An isolated nucleic acid comprising a nucleotide sequence that encodes the amino acid sequence of SEQ ID NO:2

4. An isolated nucleic acid comprising the nucleotide sequence of SEQ ID NO:1

5. An isolated nucleic acid comprising a nucleotide sequence that encodes the amino acid sequence having at least 85% identity with residues 10 through 83 of SEQ ID NO:2

6. An isolated nucleic acid comprising the nucleotide sequence having least 85% identity with nucleotides 28 through 249 of SEQ ID NO:1

7. An isolated nucleic acid comprising a nucleotide sequence that encodes the amino acid sequence residues 10 through 83 of SEQ ID NO:2

8. An isolated nucleic acid comprising nucleotides 28 through 249 of SEQ ID NO:1

9. An isolated S20 ribosomal polypeptide comprising an amino acid sequence having least 85% identity to the sequence of SEQ ID NO:2.

10. An isolated S20 ribosomal polypeptide comprising the amino acid sequence of SEQ ID NO:2.

11. An isolated S20 ribosomal polypeptide comprising an amino acid sequence having least 85% identity to residues 10 through 83 of SEQ ID NO:2.

12. An isolated S20 ribosomal polypeptide comprising residues 10 through 83 of SEQ ID NO:2

13. The isolated S20 ribosomal polypeptide of claim 11 which comprises a label.

14. The isolated S20 ribosomal polypeptide of claim 11 wherein the label is selected from the group consisting of: radiolabels, fluorescent labels, amino acid tags and biotin.

15. The isolated S20 ribosomal polypeptide of claim 13 wherein said S20 ribosomal polypeptide comprises a radiolabel.

16. The isolated S20 ribosomal polypeptide of claim 13 wherein said S20 ribosomal polypeptide comprises a fluorescent label.

17. The isolated S20 ribosomal polypeptide of claim 13 wherein said S20 ribosomal polypeptide comprises an amino acid tag.

18. The isolated S20 ribosomal polypeptide of claim 13 wherein said S20 ribosomal polypeptide comprises a biotin molecule

19. A vector comprising the nucleic acid of claim 5

20. A host cell comprising the vector of claim 19

21. A method of making isolated an S20 ribosomal polypeptide comprising:
a) introducing the nucleic acid of claim 5 into a host cell
b) maintaining said host cell under conditions whereby said nucleic acid is expressed to produce said S20 ribosomal polypeptide
c) purifying said S20 ribosomal polypeptide

22. A method for testing for inhibitors of ribosomal assembly comprising the steps of:
a) contacting the S20 ribosomal polypeptide of claim 11 with a 16S ribosomal RNA
(i) in the presence of a test agent; and
(ii) in the absence of said test agent; and
b) determining the amount of said S20 ribosomal polypeptide specifically bound to said RNA
(i) in the presence of a test agent; and
(ii) in the absence of said test agent; and
c) comparing the amount of said S20 ribosomal polypeptide determined in step (b)(i) to the amount of said S20 ribosomal polypeptide determined in step (b)(ii);

23. The method of claim 22 wherein said S20 ribosomal polypeptide comprises residues 10 through 83 of SEQ ID NO:2

24. The method of claim 22 wherein said S20 ribosomal polypeptide is labeled

25. The method of claim 22 wherein said S20 ribosomal polypeptide comprises a radiolabel

26. The method of claim 22 wherein said S20 ribosomal polypeptide comprises an amino acid tag.

27. The method of claim 22 wherein said S20 ribosomal polypeptide comprises a biotin molecule.

28. The method of claim 22 wherein said 16S ribosomal RNA comprises nucleotide position 1419 to 1502 of SEQ ID NO:21.

29. The method of claim 22 wherein said 16S ribosomal RNA comprises nucleotide position 120 to 322 of SEQ ID NO:21.

30. The method of claim 22 wherein said 16S ribosomal RNA is labeled

31. The method of claim 22 wherein said 16S ribosomal RNA comprises a radiolabel

32. The method of claim 22 wherein said 16S ribosomal RNA comprises a biotin molecule

33. The method of claim 22 wherein said S20 ribosomal polypeptide is attached to a solid support.

34. The method of claim 22 wherein said 16S ribosomal RNA is attached to a solid support

35. A method for testing for inhibitors of ribosomal assembly comprising the steps of:
Contacting at least one direct binding ribosomal polypeptide selected from the group consisting of S4, S7, S8, S15, S17 and S20 with 16S ribosomal RNA
(i) in the presence of a test agent; and
(ii) in the absence of said test agent; and
b) determining the amount of direct binding protein bound to the RNA
(i) in the presence of a test agent; and
(ii) in the absence of said test agent; and
c) comparing the amount direct binding protein determined in step (b)(i) to the amount of direct binding protein determined in step (b)(ii);

36. The method of claim 35 wherein the direct binding ribosomal proteins comprise S4, S7, S8 and S20.

37. The method of claim 35 wherein the direct binding ribosomal proteins comprise S4, S7, S8, S17 and S20

38. The method of claim 35 wherein the direct binding ribosomal proteins comprise S4, S7, S8, S17, S15 and S20.

39. The method of claim 35 wherein said direct binding ribosomal polypeptide is labeled

40. The method of claim 35 wherein said direct binding ribosomal polypeptide comprises a radiolabel

41. The method of claim 35 wherein said direct binding ribosomal polypeptide comprises an amino acid tag.

42. The method of claim 35 wherein said direct binding ribosomal polypeptide comprises a biotin molecule

43. The method of claim 35 wherein said 16S ribosomal RNA is labeled

44. The method of claim 35 wherein said 16S ribosomal RNA comprises a radiolabel

45. The method of claim 35 wherein said 16S ribosomal RNA comprises a biotin molecule

46. The method of claim 35 wherein said direct binding ribosomal polypeptide is attached to a solid support.

47. The method of claim 35 wherein said 16S ribosomal RNA is attached to a solid support

48. A method for testing for inhibitors of ribosomal assembly comprising the steps of:
a) contacting S20 ribosomal polypeptide and at least one other direct binding ribosomal polypeptide selected from the group consisting of S4, S7, S8, S15 and S17 with 16S ribosomal RNA in the
(i) in the presence of a test agent; and
(ii) in the absence of said test agent; and
b) determining the amount of S20 ribosomal polypeptide or any other direct binding protein bound to the RNA
(i) in the presence of a test agent; and
(ii) in the absence of said test agent; and
c) comparing the amount of S20 ribosomal polypeptide or any other direct binding protein determined in step (b)(i) to the amount of S20 ribosomal polypeptide or any other direct binding protein determined in step (b)(ii);

49. The method of claim 48 wherein the other direct binding ribosomal proteins comprise S4, S7, S8.

50. The method of claim 48 wherein the other direct binding ribosomal proteins comprise S4, S7, S8 and S17.

51. The method of claim 48 wherein the other direct binding ribosomal proteins comprise S4, S7, S8, S17, S15.

52. The method of claim 48 wherein said S20 ribosomal polypeptide or other direct binding ribosomal polypeptide is labeled

53. The method of claim 48 wherein said S20 ribosomal polypeptide or other direct binding ribosomal polypeptide comprises a radiolabel

54. The method of claim 48 wherein said S20 ribosomal polypeptide or other direct binding ribosomal polypeptide comprises an amino acid tag.

55. The method of claim 48 wherein said S20 ribosomal polypeptide or other direct binding ribosomal polypeptide comprises a biotin molecule

56. The method of claim 48 wherein said 16S ribosomal RNA is labeled

57. The method of claim 48 wherein said 16S ribosomal RNA comprises a radiolabel

58. The method of claim 48 wherein said 16S ribosomal RNA comprises a biotin molecule

59. The method of claim 48 wherein said S20 ribosomal polypeptide or other direct binding ribosomal polypeptide is attached to a solid support.

60. The method of claim 48 wherein said 16S ribosomal RNA is attached to a solid support

61. A method for testing for inhibitors of ribosomal assembly comprising the steps of:
a.) contacting at least one direct binding ribosomal polypeptide selected from the group consisting of S4, S7, S8, S15, S17 and S20 with 16S ribosomal RNA to form a polyribonucleotide protein complex and;
b) contacting said polyribonucleotide protein complex with at least one non-direct binding ribosomal polypeptide selected from the group consisting of S1, S2, S3, S5, S6, S9, S10, S11, S12, S13, S14, S16, S18, S19, and S21.
(i) in the presence of a test agent; and
(ii) in the absence of said test agent; and
c) determining the amount of at least one non-direct binding ribosomal polypeptide bound to the RNA
(i) in the presence of a test agent; and
(ii) in the absence of said test agent; and
d) comparing the amount of least one non direct binding ribosomal polypeptide determined in step (c)(i) to the amount of non-direct binding ribosomal polypeptide protein determined in step (c)(ii);

62. The method of claim 61 wherein the direct binding ribosomal proteins comprise S4, S7, S8.

63. The method of claim 61 wherein the direct binding ribosomal proteins comprise S4, S7, S8 and S17.

64. The method of claim 61 wherein the direct binding ribosomal proteins comprise S4, S7, S8, S17, S15.

65. The method of claim 61 wherein the direct binding ribosomal proteins comprise S4, S7, S8, S17, S15 and S20

66. The method of claim 61 wherein the non-direct binding ribosomal proteins comprise S16

67. The method of claim 61 wherein the non-direct binding ribosomal proteins comprise S3, S5, S9, S10, S12, S14, S16 and S19

68. The method of claim 61 wherein said direct binding or non-direct binding ribosomal polypeptide is labeled

69. The method of claim 61 wherein said direct binding or non-direct binding ribosomal polypeptide comprises a radiolabel

70. The method of claim 61 wherein said direct binding or non-direct binding ribosomal polypeptide comprises an amino acid tag.

71. The method of claim 61 wherein said direct binding or non-direct binding ribosomal polypeptide comprises a biotin molecule

72. The method of claim 61 wherein said 16S ribosomal RNA is labeled

73. The method of claim 61 wherein said 16S ribosomal RNA comprises a radiolabel

74. The method of claim 61 wherein said 16S ribosomal RNA comprises a biotin molecule

75. The method of claim 61 wherein said direct binding or non-direct binding ribosomal polypeptide is attached to a solid support.

76. The method of claim 61 wherein said 16S ribosomal RNA is attached to a solid support

77. A method for testing for inhibitors of ribosomal assembly comprising the steps of:
a.) contacting S4, S7, S8, S17 and S20 ribosomal polypeptides with 16S ribosomal RNA to form a polyribonucleotide protein complex and;
b) contacting said polyribonucleotide protein complex with non-direct binding ribosomal polypeptides S3, S5, S9, S10, S12, S14, S16 and S19 to form a resultant polyribonucleotide protein complex
(iii) in the presence of a test agent; and
(iv) in the absence of said test agent; and
d) contacting non-direct binding ribosomal polypeptide S3 with said resultant polyribonucleotide protein complex;
and determining the amount of said non-direct binding ribosomal polypeptide S3 bound to said resultant polyribonucleotide protein complex;
(i) formed in the presence of said test agent; and
(ii) formed in the absence of said test agent; and
e) comparing the amount of S3 determined in step (d)(i) to the amount of S3 determined in step (d)(ii)

78. The method of claim 77 wherein said non-direct binding ribosomal polypeptide S3 is labeled.

79. The method of claim 78 wherein said non-direct binding ribosomal polypeptide S3 is radiolabeled


SEQUENCE LISTING (62 sequences)

SEQ ID NO: 1 - DNA, 252 bp, Staphylococcus aureus
atggcaaatatcaaatctgcaattaaacgtgtaaaaacaactgaaaaagctgaagcacgcaacatttcacaaaagagtgcaatgcgtacagcagttaaaaacgctaaaacagctgtttcaaataacgctgataataaaaatgaattagtaagcttagcagttaagttagtagacaaagctgctcaaagtaatttaatacattcaaacaaagctgaccgtattaaatcacaattaatgactgcaaataaataa

SEQ ID NO: 2 - protein, 83 aa, Staphylococcus aureus
MANIKSAIKRVKTTEKAEARNISQKSAMRTAVKNAKTAVSNNADNKNELVSLAVKLVDKAAQSNLIHSNKADRIKSQLMTANK

SEQ ID NO: 3 - DNA, 23 bp, Artificial Sequence
  Description of Artificial SequenceForward Sequencing Primer
aatatcaaatctgcaattaaacg

SEQ ID NO: 4 - DNA, 23 bp, Artificial Sequence
  Description of Artificial SequenceForward Sequencing Primer
aaattttgataagatgaactcac

SEQ ID NO: 5 - DNA, 22 bp, Artificial Sequence
  Description of Artificial Sequence Forward Sequencing Primer
tttaggaggtgacagaaatggc

SEQ ID NO: 6 - DNA, 24 bp, Artificial Sequence
  Description of Artificial SequenceForward Sequencing Primer
acgcaacatttcacaaaagagtgc

SEQ ID NO: 7 - DNA, 25 bp, Artificial Sequence
  Description of Artificial SequenceReverse Sequencing Primer
attgcactcttttgtgaaatgttgc

SEQ ID NO: 8 - DNA, 23 bp, Artificial Sequence
  Description of Artificial SequenceReverse Sequencing Primer
atctttataaaaaataaaagttc

SEQ ID NO: 9 - DNA, 41 bp, Artificial Sequence
  Description of Artificial SequencePCR Primer
gtgttatcgataatggcaaatatcaaatctgcaattaaacg

SEQ ID NO: 10 - DNA, 37 bp, Artificial Sequence
  Description of Artificial SequencePCR Primer
gtgttggatccttatttatttgcagtcattaattgtg

SEQ ID NO: 11 - DNA, 694 bp, Staphylococcus aureus
aacactcttttttgtttattcataacaacaaaaaagaattaaaggaggagtcttattatggctcgattcagaggttcaaactggaaaaaatctcgtcgtttaggtatctctttaagcggtactggtaaagaattagaaaaacgtccttacgcaccaggacaacatggtccaaaccaacgtaaaaaattatcagaatatggtttacaattacgtgaaaaacaaaaattacgttacttatatggaatgactgaaagacaattccgtaacacatttgacatcgctggtaaaaaattcggtgtacacggtgaaaacttcatgatcttattagcaagtcgtttagacgctgttgtttattcattaggtttagctcgtactcgtcgtcaagcacgtcaattagttaaccacggtcatatcttagtagatggtaaacgtgttgatattccatcttattctgttaaacctggtcaaacaatttcagttcgtgaaaaatctcaaaaattaaacatcatcgttgaatcagttgaaatcaacaatttcgtacctgagtacttaaactttgatgctgacagcttaactggtactttcgtacgtttaccagaacgtagcgaattacctgctgaaattaacgaacaattaatcgttgagtactactcaagataatacggtcaataccaacacccacaattgtgggtgt

SEQ ID NO: 12 - protein, 200 aa, Staphylococcus aureus
MARFRGSNWKKSRRLGISLSGTGKELEKRPYAPGQHGPNQRKKLSEYGLQLREKQKLRYLYGMTERQFRNTFDIAGKKFGVHGENFMILLASRLDAVVYSLGLARTRRQARQLVNHGHILVDGKRVDIPSYSVKPGQTISVREKSQKLNIIVESVEINNFVPEYLNFDADSLTGTFVRLPERSELPAEINEQLIREYYSR

SEQ ID NO: 13 - DNA, 667 bp, Staphylococcus aureus
ttcattatacggaactaagaaacctaaaaactaagaatttagtttttaattaaatcttaaacttaaaatatttaatataaggaagggaggatttacattatgcctcgtaaaggatcagtacctaaaagagacgtattaccagatccaattcataactctaagttagtaactaaattaattaacaaaattatgttagatggtaaacgtggaacagcacaaagaattctttattcagcattcgacctagttgaacaacgcagtggtcgtgatgcattagaagtattcgaagaagcaatcaacaacattatgccagtattagaagttaaagctcgtcgcgtaggtggttctaactatcaagtaccagtagaagttcgtccagagcgtcgtactactttaggtttacgttggttagttaactatgcacgtcttcgtggtgaaaaaacgatggaagatcgtttagctaacgaaattttagatgcagcaaataatacaggtggtgccgttaagaaacgtgaggacactcacaaaatggctgaagcaaacaaagcatttgctcactaccgttggtaagataaaagcttttaccctgagtgtgttctatattaatgaattttcattaagcgttcatgcttagggcatcgccatatctatcgtatttattcagtaa

SEQ ID NO: 14 - protein, 156 aa, Staphylococcus aureus
MPRKGSVPKRDVLPDPIHNSKLVTKLINKIMLDGKRGTAQRILYSAFDLVEQRSGRDALEVFEEAINNIMPVLEVKARRVGGSNYQVPVEVRPERRTTLGLRWLVNYARLRGEKTMEDRLANEILDAANNTGGAVKKREDTHKMAEANKAFAHYRW

SEQ ID NO: 15 - DNA, 615 bp, Staphylococcus aureus
atcgtaaatttaaattatgccgtatttgtttccgtgaattagcttacaaaggccaaatccctggcgttcgtaaagctagctggtaataaaaaagagtctgaaaggaggcaacaatcaatgacaatgacagatccaatcgcagatatgcttactcgtgtaagaaacgcaaacatggtgcgtcacgagaagttagaattacctgcatcaaatattaaaaaagaaattgctgaaatcttaaagagtgaaggtttcattaaaaatgttgaatacgtagaagatgataaacaaggtgtacttcgtttattcttaaaatatggtcaaaacgatgagcgtgttatcacaggattaaaacgtatttcaaaaccaggtttacgtgtttatgcaaaagctagcgaaatgcctaaagtattaaatggtttaggtattgcattagtatcaacttctgaaggtgtaatcactgacaaagaagcaagaaaacgtaatgttggtggagaaattatcgcatacgtttggtaataaaaaataaggaggtgccataacatgagtcgtgttggtaagaaaattattgacatccctagtgacgtaacagtaacttttgatggaaatcatgtaact

SEQ ID NO: 16 - protein, 132 aa, Staphylococcus aureus
MTMTDPIADMLTRVRNANMVRHEKLELPASNIKKEIAEILKSEGFIKNVEYVEDDKQGVLRLFLKYGQNDERVITGLKRISKPGLRVYAKASEMPKVLNGLGIALVSTSEGVITDKEARKRNVGGEIIAYVW

SEQ ID NO: 17 - DNA, 517 bp, Staphylococcus aureus
tagttatataaacaatctataccacacctttttcttagtaggtcgaatctccaacgcctaactcggattaaggagtattcaaacattttaaggaggaaattgattatggcaatttcacaagaacgtaaaaacgaaatcattaaagaataccgtgtacacgaaactgatactggttcaccagaagtacaaatcgctgtacttactgcagaaatcaacgcagtaaacgaacacttacgtacacacaaaaaagaccaccattcacgtcgtggattattaaaaatggtaggtcgtcgtagacatttattaaactacttacgtagtaaagatattcaacgttaccgtgaattaattaaatcacttggtatccgtcgttaatcttaatataacgtctttgaggttggggcatatttatgttccaaccttaatttatattaaaaaagctttttacaaatattaacatttattatatgttaagctaatattgagtgaataataaggttacaatgagataaagatg

SEQ ID NO: 18 - protein, 89 aa, Staphylococcus aureus
MAISQERKNEIIKEYRVHETDTGSPEVQIAVLTAEINAVNEHLRTHKKDHHSRRGLLKMVGRRRHLLNYLRSKDIQRYRELIKSLGIRR

SEQ ID NO: 19 - DNA, 401 bp, Staphylococcus aureus
tctaaaaactgttgctcgtgaaagagaaattgaacaaagtaaggctaatcaataattaagtaagaggaggttacaaaagtgagcgaaagaaacgatcgtaaagtttatgtaggtaaagttgtttcagacaaaatggacaagactattacagtacttgttgaaacttacaaaacacacaaattatacggtaaacgagtaaaatactctaaaaaatacaaaactcatgatgaaaacaattcagctaaattaggagacattgttaaaattcaagaaactcgtcctttatcagcaacaaaacgttttcgtttagtagagattgttgaagagtcagtaattatttaatacaagtttagagataagggaggtttaactaatgatccaacaagaaacacgcttgaaag

SEQ ID NO: 20 - protein, 87 aa, Staphylococcus aureus
MSERNDRKVYVGKVVSDKMDKTITVLVETYKTHKLYGKRVKYSKKYKTHDENNSAKLGDIVKIQETRPLSATKRFRLVEIVEESVII

SEQ ID NO: 21 - DNA, 1555 bp, Staphylococcus aureus
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtattgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 22 - DNA, 1294 bp, Staphylococcus aureus
tcttgacaattctgtcagtttataagatgttataaatatgtagtgtataaggaggcaaacaagatgactgaagaattcaatgaatcaatgattaacgatattaaagaaggtgacaaagtcactggcgaggtacaacaagttgaagacaagcaagttgttgttcatatcaacggtggtaaatttaatgggattattcctattagtcaactatctacgcatcatattgatagcccaagtgaagttgtaaaagagggcgacgaagttgaagcatatgtcactaaagttgagtttgatgaagaaaatgaaactggagcttacatcttatctagaagacaacttgaaactgagaagtcttatagttatttacaagaaaaattagataataatgaaatcatcgaagcgaaagtaacagaagtagttaaaggtggtttggttgttgatgtaggacaaagaggttttgttccggcttcactaatttcaacagacttcattgaggatttctctgtgtttgatggacaaacaattcgtattaaagttgaagaattggatcctgaaaataatagagtcattttaagccgtaaagcagttgaacaagaagaaaacgatgctaaaaaagatcaattattacaatctttaaatgaaggcgatgttattgatggtaaagtagcgcgtttaactcaatttggtgcatttatagacattggcggtgttgatggtttagtgcatgtatctgaactttctcacgaacatgttcaaacaccagaagaagtagtttcaattggtcaagatgttaaagttaaaattaaatctattgatagagatacagaacgtatttcattatcaatcaaagatacgttaccaacacctttcgaaaatattaaaggtcaattccacgaaaatgatgtcattgaaggtgtcgtagtaagattggcaaactttggtgcatttgttgaaattgcaccaggtgtacaaggacttgtacatatttctgaaattgcacacaaacacattggtacgccaggtgaagtgttagaacctggtcaacaagtaaatgttaaaatattaggtattgatgaagagaatgaaagagtatcactatctattaaagcaacattaccaaacgaagatgttgttgaaagtgatccttctacgactaaggcgtacttagaaaacgaagaagaagataatccaacaattggcgatatgattggtgataaacttaaaaatcttaaactataatttaatatttaatagtcaactccacatgtttatgattgcatgtggagtattttta

SEQ ID NO: 23 - protein, 391 aa, Staphylococcus aureus
MTEEFNESMINDIKEGDKVTGEVQQVEDKQVVVHINGGKFNGIIPISQLSTHHIDSPSEVVKEGDEVEAYVTKVEFDEENETGAYILSRRQLETEKSYSYLQEKLDNNEIIEAKVTEVVKGGLVVDVGQRGFVPASLISTDFIEDFSVFDGQTIRIKVEELDPENNRVILSRKAVEQEENDAKKDQLLQSLNEGDVIDGKVARLTQFGAFIDIGGVDGLVHVSELSHEHVQTPEEVVSIGQDVKVKIKSIDRDTERISLSIKDTLPTPFENIKGQFHENDVIEGVVVRLANFGAFVEIAPGVQGLVHISEIAHKHIGTPGEVLEPGQQVNVKILGIDEENERVSLSIKATLPNEDVVESDPSTTKAYLENEEEDNPTIGDMIGDKLKNLKL

SEQ ID NO: 24 - DNA, 924 bp, Staphylococcus aureus
  misc_feature (271)..(271): unknown
atattgtctttacaatagtttgctatggaggtaattaaccaataggaggaatttataatggcagtaatttcaatgaaacaattactagaagcgggtgttcmcttcggtcaccaaacacgtcgttggaacccaaaaatgaaaaaatatatcttcactgagagaaatggtatttatatcatcgacttacaaaaaacagtgaaaaaagtagacgaggcatacaacttcttgaaacaagtttcagaagatggtggacaagtcttattcgtagganctaaaaaacaagcacaagaatcagttaaatctgaagcagaacgtgctggtcaattctacattaaccaaagatggttaggtggattattaactaactataaaacgatctcaaaacgaatcaaacgtatttctgaaattgaaaaaatggaagaagatggtttattcgaagtattacctaaaaaagaagtagtagaacttaaaaaagaatacgaccgtttaatcaaattcttaggcggaattcgtgatatgaaatcaatgcctcaagcattattcgtagttgacccacgtaaagagcgtaatgcaattgctgaagctcgtaaattaaatattcctatcgtaggtatcgttgacactaactgtgatcctgacgaaattgactacgttatcccagcaaacgacgatgctatccgtgcggttaaattattaactgctaaaatggcagatgcaatcttagaaggtcaacaaggcgtttctaatgaagaagtagctgcagaacaaaacatcgatttagatgaaaaagaaaaatcagaagaaacagaagcaactgaagaataatcaactgttgaatctgacttagatatagtttaaatgggtgataagatattaatgcttatcaccttttttaaaaagaaaatcgaggcaaattacaaatat

SEQ ID NO: 25 - protein, 255 aa, Staphylococcus aureus
  misc_feature (15)..(15): unknown
MAVISMKQLLEAGVXFGHQTRRWNPKMKKYIFTERNGIYIIDLQKTVKKVDEAYNFLKQVSEDGGQVLFVGTKKQAQESVKSEAERAGQFYINQRWLGGLLTNYKTISKRIKRISEIEKMEEDGLFEVLPKKEVVELKKEYDRLIKFLGGIRDMKSMPQALFVVDPRKERNAIAEARKLNIPIVGIVDTNCDPDEIDYVIPANDDAIRAVKLLTAKMADAILEGQQGVSNEEVAAEQNIDLDEKEKSEETEATEE

SEQ ID NO: 26 - DNA, 800 bp, Staphylococcus aureus
aacaaacgtacaagccacattacaatcgtcgtaagtgacggtaaagaagaagctaaagaagcttaattaacttttaaggagggaatactgtgggtcaaaaaattaatccaatcggacttcgtgttggtattatccgtgattgggaagctaaatggtatgctgaaaaagacttcgcttcacttttacacgaagatttaaaaatccgtaaatttattgataatgaattaaaagaagcatcagtttctcacgtagagattgaacgtgctgcaaaccgtatcaacattgcaattcatactggtaaacctggtatggtaattggtaaaggcggttcagaaatcgaaaaattacgcaacaaattaaatgcgttaactgataaaaaagtacacatcaacgtaattgaaatcaaaaaagttgatcttgacgctcgtttagtagctgaaaacatcgcacgtcaattagaaaaccgtgcttcattccgtcgtgtacaaaaacaagcaatcactagagctatgaaacttggtgctaaaggtatcaaaactcaagtatctggtcgtttaggcggagctgacatcgctcgtgctgaacaatattcagaaggaactgttccacttcatacgttacgtgctgacatcgattatgcacacgctgaagctgacactacttacggtaaattaggcgttaaagtatggatttatcgtggagaagttcttcctactaagaacactagtggaggaggaaaataataatgttactaccaaaacgtgtaaaatatcgtcgtcaacatcgtcctaaaacaactg

SEQ ID NO: 27 - protein, 221 aa, Staphylococcus aureus
MGNTVGQKINPIGLRVGIIRDWEAKWYAEKDFASLLHEDLKIRKFIDNELKEASVSHVEIERAANRINIAIHTGKPGMVIGKGGSEIEKLRNKLNALTDKKVHINVIEIKKVDLDARLVAENIARQLENRASFRRVQKQAITRAMKLGAKGIKTQVSGRLGGADIARAEQYSEGTVPLHTLRADIDYAHAEADTTYGKLGVKVWIYRGEVLPTKNTSGGGK

SEQ ID NO: 28 - DNA, 639 bp, Staphylococcus aureus
tcacggacgtgttaaagcattagctgaagcagcaagagaaagcggattagaattttaatttaaaggagggacaaatacatggctcgtagagaagaagagacgaaagaatttgaagaacgcgttgttacaatcaaccgtgtagcaaaagttgtaaaaggtggtcgtcgtttccgtttcactgcattagttgtagttggagacaaaaatggtcgtgtaggtttcggtactggtaaagctcaagaggtaccagaagcaatcaaaaaagctgttgaagcagctaaaaaagatttagtagttgttccacgtgttgaaggtacaactccacacacaattactggccgttacggttcaggaagcgtatttatgaaaccggctgcacctggtacaggagttatcgctggtggtcctgttcgtgccgtacttgaattagcaggtatcactgatatcttaagtaaatcattaggatcaaacacaccaatcaacatggttcgtgctacaatcgatggtttacaaaaccttaaaaatgctgaagatgttgcgaaattacgtggcaaaacagtagaagaattatacaattaaggagggaaaactagttatggctaaattacaaattaccctcactcgtagtgttattggtcg

SEQ ID NO: 29 - protein, 166 aa, Staphylococcus aureus
MARREEETKEFEERVVTINRVAKVVKGGRRFRFTALVVVGDKNGRVGFGTGKAQEVPEAIKKAVEAAKKDLVVVPRVEGTTPHTITGRYGSGSVFMKPAAPGTGVIAGGPVRAVLELAGITDILSKSLGSNTPINMVRATIDGLQNLKNAEDVAKLRGKTVEELYN

SEQ ID NO: 30 - DNA, 499 bp, Staphylococcus aureus
gcgcatgatataattctttattgtgagtaatgaaaattattccttgcttatctgttttaagattgataagccgtatagaccacaaggaggtgcaaatataaaatgagaacatatgaagttatgtacatcgtacgcccaaacattgaggaagatgctaaaaaagcgttagttgaacgtttcaacggtatcttagctactgaaggtgcagaagttttagaagcaaaagactggggtaaacgtcgcctagcttatgaaatcaatgatttcaaagatggcttctacaacatcgtacgtgttaaatctgataacaacaaagctactgacgaattccaacgtctagctaaaatcagtgacgatatcattcgttacatggttattcgtgaagacgaagacaagtaataattagagggggcgtttaaatgctaaatagagttgtattagtaggtcgtttaacgaaagatccggaatacagaaccactccctcaggtgtgagtgtagc

SEQ ID NO: 31 - protein, 98 aa, Staphylococcus aureus
MRTYEVMYIVRPNIEEDAKKALVERFNGILATEGAEVLEAKDWGKRRLAYEINDFKDGFYNIVRVKSDNNKATDEFQRLAKISDDIIRYMVIREDEDK

SEQ ID NO: 32 - DNA, 462 bp, Staphylococcus aureus
gtgcacaacaaccagaaaactacgaattacgtggttaattagaaggaggaaatgactttggcacaagttgaatatagaggcacaggccgtcgtaaaaactcagtagcacgtgtacgtttagtaccaggtgaaggtaacatcacagttaataaccgtgacgtacgcgaatacttaccattcgaatcattaattttagacttaaaccaaccatttgatgtaactgaaactaaaggtaactatgatgttttagttaacgttcatggtggtggtttcactggacaagctcaagctatccgtcacggaatcgctcgtgcattattagaagcagatcctgaatacagaggttctttaaaacgcgctggattacttactcgtgacccacgtatgaaagaacgtaaaaaaccaggtcttaaagcagctcgtcgttcacctcaattctcaaaacgttaattgtcggacgat

SEQ ID NO: 33 - protein, 132 aa, Staphylococcus aureus
MTLAQVEYRGTGRRKNSVARVRLVPGEGNITVNNRDVREYLPFESLILDLNQPFDVTETKGNYDVLVNVHGGGFTGQAQAIRHGIARALLEADPEYRGSLKRAGLLTRDPRMKERKKPGLKAARRSPQFSKR

SEQ ID NO: 34 - DNA, 441 bp, Staphylococcus aureus
aggttactgacacacccggccgctttgccatggcgctgtgtaagatagttttcgtggagaagtctatcactaaatgtagacgaataaggagggaaaattatggcaaaacaaaaaatcagaatcagattaaaagcttatgatcaccgcgtaattgatcaatcagcagagaagattgtagaaacagcgaaacgttctggtgcagatgtttctggaccaattccgttaccaactgagaaatcacgtacacacaaacgtttaatcgatattgtaaacccaacaccaaaaacagttgacgctttaatgggcttaaacttaccatctggtgtagacatcgaaatcaaattataatagacaattttaggaggtggactttcgatgaccaaaggaatcttaggaagaaaaattgggatgacacaagtattcggagaaaacggtgaatta

SEQ ID NO: 35 - protein, 102 aa, Staphylococcus aureus
MAKQKIRIRLKAYDHRVIDQSAEKIVETAKRSGADVSGPIPLPTEKSVYTIIRAVHKYKDSREQFEQRTHKRLIDIVNPTPKTVDALMGLNLPSGVDIEIKL

SEQ ID NO: 36 - DNA, 594 bp, Staphylococcus aureus
agttcgtggtcaaaaaacgaaaaacmacgcgcgtactcgtaaaggaccagttaaaacggtagctaacaagaaaaaatmataggtaaaggaggcaaattttaaatggcacgtaaacaagtatctcgtaaacgtagagtgaaaaagaatattgaaaatggtgtagcacacatccgttcaacattcaacaacactattgtaactatcactgatgagttcggtaatgctttatcatggtcatcagctggtgcattaggattcaaaggatctaaaaaatcaacaccatttgcagcacaaatggcttctgaaactgcatctaaatcagctatggagcatggtttaaaaacagttgaagtaacagttaaaggacctggtccaggtcgtgaatcagctattcgtgcattacaatctgcaggtttagaagtaactgcgatcagagacgttactccagtacctcataacggttgtcgtccaccaaaacgtcgtcgtgtataatttatgatggtattgttacaggtcactgagcaaacattttaaattaagtcgacgtatataaggaggatatttaaatgatagaaatcgaaaaacctagaattg

SEQ ID NO: 37 - protein, 129 aa, Staphylococcus aureus
MARKQVSRKRRVKKNIENGVAHIRSTFNNTIVTITDEFGNALSWSSAGALGFKGSKKSTPFAAQMASETASKSAMEHGLKTVEVTVKGPGPGRESAIRALQSAGLEVTAIRDVTPVPHNGCRPPKRRRV

SEQ ID NO: 38 - DNA, 620 bp, Staphylococcus aureus
ttaaatgagaattagtaagtgttttacttactaaattttatttaacctaaaaatgaaccacctggatgtgtgggattaaaaagtgaagagaggaggacatatcacatgccaactattaaccaattagtacgtaaaccaagacaaagcaaaatcaaaaaatcagattctccagctttaaataaaggtttcaacagtaaaaagaaaaaatttactgacttaaactcaccacaaaaacgtggtgtatgtactcgtgtaggtacaatgacacctaaaaaacctaactcagcgttacgtaaatatgcacgtgtgcgtttatcaaacaacatcgaaattaacgcatacatccctggtatcggacataacttacaagaacacagtgttgtacttgtacgtggtggacgtgtaaaagacttaccaggtgtgcgttaccatattgtacgtggagcacttgatacttcaggtgttgacggacgtagacaaggtcgttcattatacggaactaagaaacctaaaaactaagaatttagtttttaattaaatcttaaacttaaaatatttaatataaggaagggaggatttacattatgcctcgtaaaggatcagtacctaaaagagacgta

SEQ ID NO: 39 - protein, 137 aa, Staphylococcus aureus
MPTINQLVRKPRQSKIKKSDSPALNKGFNSKKKKFTDLNSPQKRGVCTRVGTMTPKKPNSALRKYARVRLSNNIEINAYIPGIGHNLQEHSVVLVRGGRVKDLPGVRYHIVRGALDTSGVDGRRQGRSLYGTKKPKN

SEQ ID NO: 40 - DNA, 633 bp, Staphylococcus aureus
gtataaaaatgaaagtaagaccatcagtaaaacctatttgcgaaaaatgtaaagtcattaaacgtaaaggtaaagtaatggtaatttgtgaaaatccaaaacacaaacaaagacaaggttaataaaagagaggtgtaaattaatatggcacgtattgcaggagtagatattccacgtgaaaaacgcgtagttatctcattaacttatatatacggtatcggtacgtcaactgctcaaaaaattcttgaagaagctaacgtatcagctgatactcgtgtgaaagatttaactgatgacgaattaggtcgcatccgtgaagttgtagacggttataaagtcgaaggtgacttacgtcgtgaaactaacttaaatatcaaacgtttaatggaaatttcatcataccgtggtatccgtcaccgtcgtggtttaccagttcgtggtcaaaaaacgaaaaacaacgcgcgtactcgtaaaggaccagttaaaacggtagctaacaagaaaaaataataggtaaaggaggcaaattttaaatggcacgtaaacaagtatctcgtaaacgtagagtgaaaaagaatattgaaaatggtgtagcacacatccgttcaacattcaacaacactattgtaacta

SEQ ID NO: 41 - protein, 121 aa, Staphylococcus aureus
MARIAGVDIPREKRVVISLTYIYGIGTSTAQKILEEANVSADTRVKDLTDDELGRIREVVDGYKVEGDLRRETNLNIKRLMEISSYRGIRHRRGLPVRGQKTKNNARTRKGPVKTVANKKK

SEQ ID NO: 42 - DNA, 311 bp, Staphylococcus aureus
ctcgtgaattgttagctaacttcggtatgccattccgtaaataattatttaaaggaggctaattaagtggctaaaacttcaatggttgctaagcaacaaaaaaaacaaaaatatgcagttcgtgaatacactcgttgtgaacgttgtggccgtccacattctgtatatcgtaaatttaaattatgccgtatttgtttccgtgaattagcttacaaaggccaaatccctggcgttcgtaaagctagctggtaataaaaaagagtctgaaaggaggcaacaatcaatgacaatgacagatccaatcgcagata

SEQ ID NO: 43 - protein, 61 aa, Staphylococcus aureus
MAKTSMVAKQQKKQKYAVREYTRCERCGRPHSVYRKFKLCRICFRELAYKGQIPGVRKASW

SEQ ID NO: 44 - DNA, 710 bp, Staphylococcus aureus
aacattcatacacctgttaatattatttcttgtagaaaataaaaattaaaacatgacttaaaggagattttataaatggcagttaaaattcgtttaacacgtttaggttcaaaaagaaatccattctatcgtatcgtagtagcagatgctcgttctccacgtgacggacgtatcatcgaacaaatcggtacttataacccaacgagcgctaatgctccagaaattaaagttgacgaagcgttagctttaaaatggttaaatgatggtgcgaaaccaactgatacagttcacaatatcttatcaaaagaaggtattatgaaaaaatttgacgaacaaaagaaagctaagtaatttagcgtaaaattgttctaacaataagaataactcgtttacactgacagttattactcaatgatacgttgggaatatcacatgttagtaatatagaacgtttgggtaccataatggtgccctttttctttgaattattttcaattaaaatagaagtggtcaaagcatagagttggaggtaatagaatgagagttgaagttggtcaaaattgtttacacacacggggtttaaaaggtggaaattaaaggtaaatccatttcagaccttttacagaccggttcggttttcaaccccggtccaaagatgcctgaccagttgggccttaaaccaaattaaaccgacccccttggaaatatta

SEQ ID NO: 45 - protein, 92 aa, Staphylococcus aureus
MAVKIRLTRLGSKRNPFYRIIVVADARSPRDGRIIEQIGTYNPTSANAPEIKVDEALALKWLNDGAKPTDTVHNILSKEGIMKKFDEQKKAK

SEQ ID NO: 46 - DNA, 437 bp, Staphylococcus aureus
aatgcaaacggaccgattgatataagtgatgatgacttaccattctaataaaaattaacgaaattaaagcgaaaaaattatcaaaggaggcacacaatcatggcaggtggaccaagaagaggcggacgtcgtcgtaaaaaagtatgctatttcacagcaaatggtattacacatatcgactacaaagacactgaattattaaaacgttttatctcagaacgcggtaaaattttaccacgtcgtgtaactggtacttcagctaaatatcaacgtatgttgactacagctatcaaacgttctcgtcatatggcattattaccatatgttaaagaagaacaataatatataatttattgtcaaaccccgtaggcataggcttacggggctttttgtgttttggggtatagaaaaagggcaaaaaggatgatgtgaatgtt

SEQ ID NO: 47 - protein, 80 aa, Staphylococcus aureus
MAGGPRRGGRRRKKVCYFTANGITHIDYKDTELLKRFISERGKILPRRVTGTSAKYQRMLTTAIKRSRHMALLPYVKEEQ

SEQ ID NO: 48 - DNA, 478 bp, Staphylococcus aureus
aaacttatcgttcgtggacgtaagaaaaaataatataatcaacttatttgggtgtgcggcttaaagctgcacgcacataataagaagggaggcgcccaaatggctcgtagtattaaaaaaggacctttcgtcgatgagcatttaatgaaaaaagttgaagctcaagaaggaagcgaaaagaaacaagtaatcaaaacatggtcacgtcgttctacaattttccctaatttcatcggacatacttttgcagtatacgacggacgtaaacacgtacctgtatatgtaactgaagatatggtaggtcataaattaggtgagtttgctcctactcgtacattcaaaggacacgttgcagacgacaagaaaacaagaagataatatctattaagtagaggaggacatcctaatggaagcaaaagcggttgctagaacaataagaatcgcacctcgtaaagtaagactagttcttgacttaatc

SEQ ID NO: 49 - protein, 92 aa, Staphylococcus aureus
MARSIKKGPFVDEHLMKKVEAQEGSEKKQVIKTWSRRSTIFPNFIGHTFAVYDGRKHVPVYVTEDMVGHKLGEFAPTRTFKGHVADDKKTRR

SEQ ID NO: 50 - DNA, 520 bp, Staphylococcus aureus
tgcaaaattttaagctaaccccatcaaataaatgattgcacaacggttagacttttgttaaaatatttcttgttgtaatcaaataaaattttgataagatgaactcacttttaggaggtggcagaaatggcaaatatcaaatctgcaattaaacgtgtaaaaacaactgaaaaagctgaagcacgcaacatttcacaaaagagtgcaatgcgtacagcagttaaaaacgctaaaacagctgtttcaaataacgctgataataaaaatgaattagtaagcttagcagttaagttagtagacaaagctgctcaaagtaatttaatacattcaaacaaagctgaccgtattaaatcacaattaatgactgcaaataaataatctttttaaataaaagttcaagcgcatgcttgaacttttattttttataaagatagaatgaataattccagtattaactgtttatccatatatgatgatttaagtttataatcagtttccgcacaagcatctataatattca

SEQ ID NO: 51 - DNA, 499 bp, Staphylococcus aureus
tgtttcaaataaaaaacaatttactaattgaccataaattacagatatattatacttataaatgcatagttttactgtgcaattgactataaagttccgttgatatttggagggagggaaatacagatgtctaaaacagtagtacgtaaaaatgaatcacttgaagatgcgttacgtagatttaaacgttcagtttctaaaagtggaacaatccaagaagtacgtaaacgtgaattttacgaaaaaccaagcgtaaaacgtaaaaagaaatcagaagctgcacgtaaacgtaaattcaaataattaatacctctgttgactccctcaacacgaatattaattatataaaacaaacatcacaagttagtgtctgacactaatatgtgatgtttttttgttgtcaatttttaattaaaaaaagttatatagtttataaataatcaaattgatattctataggttcttataactataaagtatattcaatttcatgtataat

SEQ ID NO: 52 - protein, 58 aa, Staphylococcus aureus
MSKTVVRKNESLEDALRRFKRSVSKSGTIQEVRKREFYEKPSVKRKKKSEAARKRKFK

SEQ ID NO: 53 - DNA, 31 bp, Artificial Sequence
  Description of Artificial SequencePCR Primer
tatattatcgataatggctcgattcagaggt

SEQ ID NO: 54 - DNA, 36 bp, Artificial Sequence
  Description of Artificial SequencePCR Primer
tataggatccttaacggattaattgttcgttaattt

SEQ ID NO: 55 - DNA, 33 bp, Artificial Sequence
  Description of Artificial SequencePCR Primer
tatattatcgataatggcaggtggaccaagaag

SEQ ID NO: 56 - DNA, 30 bp, Artificial Sequence
  Description of Artificial SequencePCR Primer
tataggatccttattgttcttctttaacat

SEQ ID NO: 57 - DNA, 35 bp, Artificial Sequence
  Description of Artificial SequencePCR Primer
tatattatcgataatgaagaaacatatgaagttat

SEQ ID NO: 58 - DNA, 30 bp, Artificial Sequence
  Description of Artificial SequencePCR Primer
tataggatccttacttgtcttcgtcttcac

SEQ ID NO: 59 - DNA, 19 bp, Artificial Sequence
  Description of Artificial SequencePCR Primer
caccacgagagtttgtaac

SEQ ID NO: 60 - DNA, 21 bp, Artificial Sequence
  Description of Artificial SequencePCR Primer
caccccaatcatttgtcccac

SEQ ID NO: 61 - DNA, 19 bp, Artificial Sequence
  Description of Artificial SequencePCR Primer
cacgtggataacctaccta

SEQ ID NO: 62 - DNA, 21 bp, Artificial Sequence
  Description of Artificial SequencePCR Primer
gtggccgatcaccctctcagg